(19) United States
(12) Patent Application Publication
(10) Pub. No.: US 2016/0159552 A1
(43) Pub. Date: Jun. 9, 2016

(54) PRODUCTS FOR THE OXIDATIVE COLOUR CHANGING OF KERATIN FIBRES IN A DISPENSER

(71) Applicant: Henkel AG & Co. KGaA, Duesseldorf (DE)

(72) Inventors: Constanze Neuba, Grevenbroich (DE); Norbert Schettiger, Hilden (DE); Burkhard Mueller, Duesseldorf (DE)

(21) Appl. No.: 15/040,115

(22) Filed: Feb. 10, 2016

Related U.S. Application Data
(63) Continuation of application No. PCT/DE2014/200420, filed on Aug. 22, 2014.

(30) Foreign Application Priority Data
Aug. 27, 2013 (DE) 10 2013 217 026.2

(51) Int. Cl. B65D 81/32; A61K 8/72; A61Q 5/10

(52) U.S. Cl. CPC B65D 81/32; A61Q 5/10; A61K 8/72; A61K 2800/882

(57) ABSTRACT

A product for the oxidative coloring and/or lightening of keratinic fibers, includes: 1) a dispenser, which has two chambers (A) and (B) separate from one another, and has an outlet opening (C), which is in communication with chamber (A) and with chamber (B), 2) a preparation (a) in chamber (A), including in an aqueous cosmetic carrier one or more thickening polymers in a total amount by weight (G1) of 0.1 to 40% by weight, based on the total weight of preparation (a), 3) a preparation (b) in chamber (B), including in an aqueous cosmetic carrier one or more thickening polymers in a total amount by weight (G2) of 0.1 to 40% by weight, based on the total weight of preparation (b). The weight ratio G1/G2 has a value of 0.3 to 3.0.

### FIELD OF THE INVENTION

The present invention generally relates to a product for the coloring and/or lightening of keratinic fibers, the product include a dispenser with two chambers separate from one another, which have a common outlet opening, whereby each of the two chambers in each case includes a preparation with thickening polymers in special weight ratios. Furthermore, the present invention relates a method in which keratinic fibers are colored and/or lightened with the use of said product.

### BACKGROUND OF THE INVENTION

The changing of the color of keratinic fibers, particularly of hair, is an important field in modern cosmetics. As a result, the appearance of the hair can be adapted both to the current fashion trend and to the individual wishes of the individual person. The skilled artisan is aware of various options for changing the color of hair.

Hair color can be changed temporarily by the use of direct dyes. In this case, already formed dyes diffuse from the coloring agent into the hair fiber. Dyeing with direct dyes is associated with less hair damage, but a disadvantage is the low durability and the rapid washing out of the colors obtained with direct dyes.

Oxidative color-changing agents are usually used if the consumer wants a long-lasting color result or a shade that is lighter than the consumer's original hair color. So-called oxidation coloring agents are used for permanent, intensive colors with suitable fastness properties. Such coloring agents typically include oxidation dye precursors, so-called developer components and coupler components, which together form the actual dyes under the influence of oxidizing agents, usually hydrogen peroxide. Oxidation coloring agents are characterized by excellent, long-lasting color results.

The mere lightening or bleaching of hair often occurs with the use of oxidizing agents without the addition of oxidation dye precursors. The use of hydrogen peroxide alone as an oxidizing agent is sufficient for an average bleaching effect; a mixture of hydrogen peroxide and peroxydisulfate salts is generally employed to achieve a greater bleaching effect.

Oxidative color-changing agents are typically found on the market in the form of two-component agents, in which the two components are packaged separately in two separate containers and must be mixed together shortly before use.

The first component is usually a formulation, which has been made alkaline and is often available in the form of a cream or a gel and which, provided that a change in color as well is desired concurrently with the lightening, also includes in addition oxidation dye precursors. This first component is provided in most cases in a tube, less often in a plastic or glass container.

The second component is a formulation, which has usually been made acidic for reasons of stability and which includes hydrogen peroxide in concentrations of 3 to 12% by weight as the oxidizing agent. The oxidizing agent formulation mostly has the form of an emulsion or dispersion and as a rule is made available in a plastic bottle with a reclosable outlet opening (developer bottle).

To prepare the ready-to-use mixture, the consumer must mix the two components together shortly before use. For this purpose, normally the alkaline cream or gel component must be transferred completely from the tube or glass or plastic container into the developer bottle; then both components are mixed together as completely and homogeneously as possible by shaking, and they are finally removed via an outlet opening in the top of the developer bottler.

This separate mixing operation has a number of drawbacks for consumers, however. Thus, the quantity ratio of the two components can be changed by the incomplete emptying of the tube; this results in variations in the desired color result. If the shaking or mixing of the two components is too short, the application mixture is not homogeneous with the result of a nonuniform color result. Moreover, it is also desirable for reasons of ease of use to eliminate this mixing step entirely. To avoid these drawbacks, multi-chamber containers with a common discharge opening were developed, in which the two components are mixed in the valve or the dispenser during the discharging. The removing of the application mixture via the dispenser makes the mixing of the components by the consumer superfluous and has greatly increased the ease of use.

But with these dispensers as well, there still is the problem that the mixing ratio of the two components can change depending on the filling level of the formulations in the chambers, the flow behavior of the two formulations, and depending on the pressure within the dispenser. This poses the great risk that the composition of the application mixture changes during its removal. The associated variations in the color result are very greatly not desired by the consumer.

It has not been possible so far to provide oxidative hair color changing products that are based on a multi-chamber system and enable dosing of the application mixture with a constant, defined composition. The attempt was made in DE 10 2007 056 935 A1 to assure the constancy of the mixing ratio by developing dispensers with a special control mechanism. This mechanism, however, does not rule out incorrect operation on the part of the consumer.

It is therefore desirable to provide a new product for the oxidative coloring and/or lightening of keratinic fibers, which product is based on a dispenser that enables the removal of the finished application mixture. In this case, the application mixture removed via the dispenser should be defined and constant with respect to its composition and not change as a function of the degree of dispenser filling.

Furthermore, other desirable features and characteristics of the present invention will become apparent from the subsequent detailed description of the invention and the appended claims, taken in conjunction with the accompanying drawings and this background of the invention.

### Brief Summary of the Invention

A product for the oxidative coloring and/or lightening of keratinic fibers includes (1) a dispenser, which has two chambers (A) and (B) separate from one another, has an outlet opening (C), which is in communication with chamber (A) and with chamber (B); (2) a preparation (a) in chamber (A), including in an aqueous cosmetic carrier one or more thickening polymers in a total amount by weight (G1) of 0.1 to 40% by weight, based on the total weight of preparation (a); and (3) a preparation (b) in chamber (B), including in an aqueous cosmetic carrier one or more thickening polymers in a total amount by weight (G2) of 0.1 to 40% by weight, based on the total weight of preparation (b), characterized in that the weight ratio G1/G2 has a value of 0.3 to 3.0.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

Surprisingly, it was found by the present inventors that it is possible to obtain an application mixture of invariable composition from a multi-chamber dispenser, if the two preparations in the multi-chamber dispenser are precisely matched with respect to their content and composition of thickening polymers.

A first aspect of the invention is a product for the oxidative coloring and/or lightening of keratinic fibers, including

(1) a dispenser, which

has two chambers (A) and (B) separate from one another,

has an outlet opening (C), which is in communication with chamber (A) and with chamber (B),

(2) a preparation (a) in chamber (A), including in an aqueous cosmetic carrier

one or more thickening polymers in a total amount by weight (G1) of 0.1 to 40% by weight, based on the total weight of preparation (a),

(3) a preparation (b) in chamber (B), including in an aqueous cosmetic carrier

one or more thickening polymers in a total amount by weight (G2) of 0.1 to 40% by weight, based on the total weight of preparation (b),

characterized in that

the weight ratio G1/G2 has a value of 0.3 to 3.0.

Keratinic fibers, keratin-including fibers, or keratin fibers are to be understood to mean pelts, wool, feathers, and in particular human hair. Although the agents of the invention are primarily suitable for lightening and coloring keratin fibers, there is nothing which in principle precludes use in other fields as well.

Preparations (a) and (b) located in chambers (A) and (B) include the essential ingredients in each case in a cosmetic carrier, preferably in a suitable aqueous, alcoholic, or aqueous-alcoholic carrier. For the purpose of oxidative color changing, such carriers can be, for example, creams, emulsions, gels, or surfactant-including foaming solutions as well, such as, for example, shampoos, foam aerosols, foam formulations, or other preparations suitable for use on hair. Preparations (a) and/or (b) are especially preferably gels.

Their precisely matched content of thickening polymers is characteristic for preparations (a) and (b). “Thickening polymers” within the meaning of the present invention are understood to mean organic polymeric compounds that increase the viscosity of the water, if they are added in an amount of 0.1% by weight to water (at 22° C. and a pressure of 760 mm Hg). The measurement of the viscosity occurs in this case at 22° C. (22° C./Brookfield viscometer/spindle 5/4 rpm).

Polymeric compounds within the meaning of the present invention are understood to mean compounds, based on repeat units, with a molar weight of more than 5000 g/mol, preferably of more than 10,000 g/mol, and particularly preferably of more than 20,000 g/mol. The maximum molar weight of the polymers depends on the synthesis conditions, batch size, and the degree of polymerization and preferably does not exceed 108 g/mol.

Compounds based on repeat units have at least 50 repeat units, preferably at least 100 repeat units, and particularly preferably at least 500 repeat units.

Preparation (a), which is located in chamber (A) of the dispenser, includes in a cosmetic carrier one or more thickening polymers in a total amount by weight (G1) of 0.1 to 40% by weight, based on the total weight of preparation (a). Preparation (b), which is located in chamber (B) of the dispenser, includes in a cosmetic carrier one or more thickening polymers in a total amount by weight (G2) of 0.1 to 40% by weight, based on the total weight of preparation (b). In this respect, a feature, essential to the invention, of the product is that the weight ratio G1/G2 has a value of 0.3 to 3.0.

The total amount by weight G1 is understood to mean the total weight of all thickening polymers included in formulation (a). The calculation basis in this case is the total weight of preparation (a).

The total amount by weight G2 is understood to mean the total weight of all thickening polymers included in formulation (b). The calculation basis in this case is the total weight of preparation (b).

If the weight ratio G1/G2 of the thickening polymers in formulations (a) and (b) is within the value range of 0.3 to 3.0, both formulations can be dispensed reproducibly and constantly and via outlet opening of dispenser (C); i.e., the removal of defined and constant amounts of formulations (a) and (b) over the entire application period is possible. An application mixture, prepared from (a) and (b) and having the same composition in each removal step, can be removed in this way via the dispenser, regardless of whether the dispenser is still completely filled or has already been partially emptied. In this regard, the dispensing is the more precise, the closer the weight ratio G1/G2 is to the value of 1.

A very especially preferred product for the oxidative coloring and/or lightening of keratinic fibers is therefore characterized in that the weight ratio of G1/G2 has a value of 0.5 to 2.0, preferably of 0.55 to 1.8, more preferably of 0.6 to 1.6, and particularly preferably of 0.7 to 1.4.

Preferably preparations (a) and (b) include thickening polymers from the group include polysaccharides, acrylic acid polymers, acrylic acid copolymers, methacrylic acid polymers, and/or methacrylic acid copolymers. If preparations (a) and (b) both include one or more thickening polymers from said group, the viscosity of both preparations can be matched especially well to one another. In particular, the viscosity of both preparations remains stable for rather long storage periods as well.

In a further especially preferred embodiment, a product of the invention is therefore characterized in that
preparation (a) includes as thickening polymer(s) one or more polymers from the group include polysaccharides, acrylic acid polymers, acrylic acid copolymers, methacrylic acid polymers, and/or methacrylic acid copolymers and
preparation (b) includes as thickening polymer(s) one or more polymers from the group include polysaccharides, acrylic acid polymers, acrylic acid copolymers, methacrylic acid polymers, and/or methacrylic acid copolymers.

It has emerged surprisingly that the rheological properties of preparations (a) and (b) can be matched especially well to one another by the use of special polysaccharides. In other words, preparations (a) and (b), which both include at least one special polysaccharide, whereby the total weight ratio of the thickening polymers G1/G2 has a value of 0.3 to 3.0, can be removed from the dispenser in an especially constant composition over the entire application period. Therefore, both preparations (a) and (b), although they can otherwise differ greatly from one another with respect to their pH and their ingredients, if anionic polysaccharides are added, have virtually identical rheological properties, which depend in particular also on the pressure within the dispenser and on the degree of dispenser filling.

The term polysaccharides is the collective term for macromolecular carbohydrates whose molecules consist of a large number of glycosidically linked monosaccharide molecules (with a molar mass of at least 5000 g/mol).

The xanthan gums, alginates, carboxyalkylcelluloses, and hyaluronic acids have a very particular suitability within the group of the polysaccacharides.

Xanthan gum is a polysaccharide, which is made up of, inter alia, the structural components: D-glucose, D-mannose, D-glucuronic acid, acetate, and pyruvate, and is also known under the INCI name Xanthan Gum. Xanthan gum carries carboxy groups and is anionic or anionizable. The physiologically acceptable salts of xanthan gum are also novel.

Salts of alginic acid are called alginates (INCI name Algin). Alginates are acidic, carboxy group-including polysaccharides, consisting of D-mannuronic acid and D-guluronic acid in different ratios, which are linked by 1-4-glycosidic bonds. The alkaline and alkaline earth salts of alginic acids in particular are novel. The use of alginic acid, sodium alginate, potassium alginate, ammonium alginate, and/or calcium alginate has proven especially advantageous in the preparations of the invention.

Carboxyalkylcelluloses are cellulose ethers in which the hydrogen atoms of the hydroxy groups of the cellulose are partially or completely substituted by carboxyalkyl groups. A preferred carboxyalkylcellulose is carboxymethylcellulose, which preferably can be used as an anionic polymer in the form of its sodium salt (sodium carboxymethylcellulose).

The basic structural unit of hyaluronic acid (INCI names: Hyaluronic acid, Sodium Hyaluronate) is an amino disaccharide which is made up of D-glucuronic acid and N-acetylglucosamine in a 1-3-glycosidic bond and is connected to the next unit with a β-1-4-glycosidic bond. The sodium and potassium salts of hyaluronic acid have also proven especially suitable within the scope of work leading to this invention.

An especially preferred product for the oxidative coloring and/or lightening of keratinic fibers is therefore characterized furthermore in that
preparation (a) includes as thickening polymer(s) one or more polysaccharides from the group include carboxymethylcelluloses, alginic acids, xanthan gum, and/or the physiologically acceptable salts thereof, especially preferably xanthan gum and/or the physiologically acceptable salts thereof, and
preparation (b) includes as thickening polymer(s) one or more polysaccharides from the group include carboxymethylcelluloses, alginic acids, xanthan gum, and/or the physiologically acceptable salts thereof, especially preferably xanthan gum and/or the physiologically acceptable salts thereof.

Especially preferred is a product for the oxidative coloring and/or lightening of keratinic fibers, include

(1) a dispenser, which

has two chambers (A) and (B) separate from one another,

has an outlet opening (C), which is in communication with chamber (A) and with chamber (B),

(2) a preparation (a) in chamber (A), including in an aqueous cosmetic carrier

one or more thickening polymers in a total amount by weight (G1) of 0.1 to 40% by weight, based on the total weight of preparation (a),

(3) a preparation (b) in chamber (B), including in an aqueous cosmetic carrier

one or more thickening polymers in a total amount by weight (G2) of 0.1 to 40% by weight, based on the total weight of preparation (b),

characterized in that

preparation (a) includes as thickening polymer(s) one or more polysaccharides from the group include carboxymethylcelluloses, alginic acids, xanthan gum, and/or the physiologically acceptable salts thereof, especially preferably xanthan gum and/or the physiologically acceptable salts thereof,

preparation (b) includes as thickening polymer(s) one or more polysaccharides from the group include carboxymethylcelluloses, alginic acids, xanthan gum, and/or the physiologically acceptable salts thereof, especially preferably xanthan gum and/or the physiologically acceptable salts thereof, and

the weight ratio G1/G2 has a value of 0.5 to 2.0.

Especially preferred is a product for the oxidative coloring and/or lightening of keratinic fibers, include

(1) a dispenser, which

has two chambers (A) and (B) separate from one another,

has an outlet opening (C), which is in communication with chamber (A) and with chamber (B),

(2) a preparation (a) in chamber (A), including in an aqueous cosmetic carrier

one or more thickening polymers in a total amount by weight (G1) of 0.1 to 40% by weight, based on the total weight of preparation (a),

(3) a preparation (b) in chamber (B), including in an aqueous cosmetic carrier

one or more thickening polymers in a total amount by weight (G2) of 0.1 to 40% by weight, based on the total weight of preparation (b), characterized in that

preparation (a) includes as thickening polymer(s) one or more polysaccharides from the group include carboxymethylcelluloses, alginic acids, xanthan gum, and/or the physiologically acceptable salts thereof, especially preferably xanthan gum and/or the physiologically acceptable salts thereof,

preparation (b) includes as thickening polymer(s) one or more polysaccharides from the group include carboxymethylcelluloses, alginic acids, xanthan gum, and/or the physiologically acceptable salts thereof, especially preferably xanthan gum and/or the physiologically acceptable salts thereof, and

the weight ratio G1/G2 has a value of 0.55 to 1.8.

Especially preferred is a product for the oxidative coloring and/or lightening of keratinic fibers, include

(1) a dispenser, which

has two chambers (A) and (B) separate from one another,

has an outlet opening (C), which is in communication with chamber (A) and with chamber (B),

(2) a preparation (a) in chamber (A), including in an aqueous cosmetic carrier

one or more thickening polymers in a total amount by weight (G1) of 0.1 to 40% by weight, based on the total weight of preparation (a),

(3) a preparation (b) in chamber (B), including in an aqueous cosmetic carrier

one or more thickening polymers in a total amount by weight (G2) of 0.1 to 40% by weight, based on the total weight of preparation (b), characterized in that

preparation (a) includes as thickening polymer(s) one or more polysaccharides from the group include carboxymethylcelluloses, alginic acids, xanthan gum, and/or the physiologically acceptable salts thereof, especially preferably xanthan gum and/or the physiologically acceptable salts thereof,

preparation (b) includes as thickening polymer(s) one or more polysaccharides from the group include carboxymethylcelluloses, alginic acids, xanthan gum, and/or the physiologically acceptable salts thereof, especially preferably xanthan gum and/or the physiologically acceptable salts thereof, and

the weight ratio G1/G2 has a value of 0.6 to 1.6.

Especially preferred is a product for the oxidative coloring and/or lightening of keratinic fibers, include

(1) a dispenser, which

has two chambers (A) and (B) separate from one another,

has an outlet opening (C), which is in communication with chamber (A) and with chamber (B),

(2) a preparation (a) in chamber (A), including in an aqueous cosmetic carrier

one or more thickening polymers in a total amount by weight (G1) of 0.1 to 40% by weight, based on the total weight of preparation (a),

(3) a preparation (b) in chamber (B), including in an aqueous cosmetic carrier

one or more thickening polymers in a total amount by weight (G2) of 0.1 to 40% by weight, based on the total weight of preparation (b),

characterized in that

preparation (a) includes as thickening polymer(s) one or more polysaccharides from the group include carboxymethylcelluloses, alginic acids, xanthan gum, and/or the physiologically acceptable salts thereof, especially preferably xanthan gum and/or the physiologically acceptable salts thereof,

preparation (b) includes as thickening polymer(s) one or more polysaccharides from the group include carboxymethylcelluloses, alginic acids, xanthan gum, and/or the physiologically acceptable salts thereof, especially preferably xanthan gum and/or the physiologically acceptable salts thereof, and

the weight ratio G1/G2 has a value of 0.7 to 1.4.

Apart from the aforementioned especially preferred polysaccharides, preparations (a) and (b) can also include in addition other thickening polymers. The requirement applies here as well, however, that the total amount by weight of all thickening polymers (G1) included in preparation (a) is 0.1 to 40% by weight and the total amount by weight of all thickening polymers (G2) included in preparation (b) is 0.1 to 40% by weight.

In another embodiment, it can be especially preferable, if preparations (a) and (b) include one or more thickening polymers from the group include carboxymethylcelluloses, alginic acids, and xanthan gum and, moreover, include no other thickening polymer.

Also preferred is a product for the oxidative coloring and/or lightening of keratinic fibers, include

(1) a dispenser, which

has two chambers (A) and (B) separate from one another,

has an outlet opening (C), which is in communication with chamber (A) and with chamber (B),

(2) a preparation (a) in chamber (A), including in an aqueous cosmetic carrier

one or more thickening polymers in a total amount by weight (G1) of 0.1 to 40% by weight, based on the total weight of preparation (a),

(3) a preparation (b) in chamber (B), including in an aqueous cosmetic carrier

one or more thickening polymers in a total amount by weight (G2) of 0.1 to 40% by weight, based on the total weight of preparation (b), characterized in that

the weight ratio G1/G2 has a value of 0.3 to 3.0, preferably of 0.5 to 2.0, more preferably of 0.55 to 1.8, even more preferably of 0.6 to 1.6, and particularly preferably in 0.7 to 1.4,

preparation (a) includes as the thickening polymer carboxymethylcellulose and/or the physiologically acceptable salt thereof and

preparation (b) includes as the thickening polymer carboxymethylcellulose and/or the physiologically acceptable salt thereof.

Also preferred is a product for the oxidative coloring and/or lightening of keratinic fibers, include

(1) a dispenser, which

has two chambers (A) and (B) separate from one another,

has an outlet opening (C), which is in communication with chamber (A) and with chamber (B),

(2) a preparation (a) in chamber (A), including in an aqueous cosmetic carrier

one or more thickening polymers in a total amount by weight (G1) of 0.1 to 40% by weight, based on the total weight of preparation (a),

(3) a preparation (b) in chamber (B), including in an aqueous cosmetic carrier

one or more thickening polymers in a total amount by weight (G2) of 0.1 to 40% by weight, based on the total weight of preparation (b),

characterized in that

the weight ratio G1/G2 has a value of 0.3 to 3.0, preferably of 0.5 to 2.0, more preferably of 0.55 to 1.8, even more preferably of 0.6 to 1.6, and particularly preferably in 0.7 to 1.4,

preparation (a) includes as the thickening polymer alginic acid and/or the physiologically acceptable salt thereof and

preparation (b) includes as the thickening polymer alginic acid and/or the physiologically acceptable salt thereof.

Also preferred is a product for the oxidative coloring and/or lightening of keratinic fibers, include

(1) a dispenser, which

has two chambers (A) and (B) separate from one another,

has an outlet opening (C), which is in communication with chamber (A) and with chamber (B),

(2) a preparation (a) in chamber (A), including in an aqueous cosmetic carrier

one or more thickening polymers in a total amount by weight (G1) of 0.1 to 40% by weight, based on the total weight of preparation (a),

(3) a preparation (b) in chamber (B), including in an aqueous cosmetic carrier

one or more thickening polymers in a total amount by weight (G2) of 0.1 to 40% by weight, based on the total weight of preparation (b),

characterized in that

the weight ratio G1/G2 has a value of 0.3 to 3.0, preferably of 0.5 to 2.0, more preferably of 0.55 to 1.8, even more preferably of 0.6 to 1.6, and particularly preferably in 0.7 to 1.4,

preparation (a) includes as the thickening polymer xanthan gum and/or the physiologically acceptable salt thereof and

preparation (b) includes as the thickening polymer xanthan gum and/or the physiologically acceptable salt thereof.

Also preferred is a product for the oxidative coloring and/or lightening of keratinic fibers, include

(1) a dispenser, which

has two chambers (A) and (B) separate from one another,

has an outlet opening (C), which is in communication with chamber (A) and with chamber (B),

(2) a preparation (a) in chamber (A), including in an aqueous cosmetic carrier

one or more thickening polymers in a total amount by weight (G1) of 0.1 to 40% by weight, based on the total weight of preparation (a),

(3) a preparation (b) in chamber (B), including in an aqueous cosmetic carrier

one or more thickening polymers in a total amount by weight (G2) of 0.1 to 40% by weight, based on the total weight of preparation (b),

characterized in that

the weight ratio G1/G2 has a value of 0.3 to 3.0, preferably of 0.5 to 2.0, more preferably of 0.55 to 1.8, even more preferably of 0.6 to 1.6, and particularly preferably in 0.7 to 1.4,

preparation (a) includes as the thickening polymer carboxymethylcellulose and/or the physiologically acceptable salt thereof,

preparation (b) includes as the thickening polymer carboxymethylcellulose and/or the physiologically acceptable salt thereof and

and preparations (a) and (b) include no other thickening polymers apart from carboxymethylcellulose and/or the physiologically acceptable salt thereof.

Also preferred is a product for the oxidative coloring and/or lightening of keratinic fibers, include

(1) a dispenser, which

has two chambers (A) and (B) separate from one another,

has an outlet opening (C), which is in communication with chamber (A) and with chamber (B),

(2) a preparation (a) in chamber (A), including in an aqueous cosmetic carrier

one or more thickening polymers in a total amount by weight (G1) of 0.1 to 40% by weight, based on the total weight of preparation (a),

(3) a preparation (b) in chamber (B), including in an aqueous cosmetic carrier

one or more thickening polymers in a total amount by weight (G2) of 0.1 to 40% by weight, based on the total weight of preparation (b),

characterized in that

the weight ratio G1/G2 has a value of 0.3 to 3.0, preferably of 0.5 to 2.0, more preferably of 0.55 to 1.8, even more preferably of 0.6 to 1.6, and particularly preferably in 0.7 to 1.4,

preparation (a) includes as the thickening polymer alginic acid and/or the physiologically acceptable salt thereof,

preparation (b) includes as the thickening polymer alginic acid and/or the physiologically acceptable salt thereof and

and preparations (a) and (b) include no other thickening polymers apart from alginic acid and/or the physiologically acceptable salt thereof.

Also preferred is a product for the oxidative coloring and/or lightening of keratinic fibers, include

(1) a dispenser, which

has two chambers (A) and (B) separate from one another,

has an outlet opening (C), which is in communication with chamber (A) and with chamber (B),

(2) a preparation (a) in chamber (A), including in an aqueous cosmetic carrier

one or more thickening polymers in a total amount by weight (G1) of 0.1 to 40% by weight, based on the total weight of preparation (a),

(3) a preparation (b) in chamber (B), including in an aqueous cosmetic carrier

one or more thickening polymers in a total amount by weight (G2) of 0.1 to 40% by weight, based on the total weight of preparation (b),

characterized in that

the weight ratio G1/G2 has a value of 0.3 to 3.0, preferably of 0.5 to 2.0, more preferably of 0.55 to 1.8, even more preferably of 0.6 to 1.6, and particularly preferably in 0.7 to 1.4,

preparation (a) includes as the thickening polymer xanthan gum and/or the physiologically acceptable salt thereof,

preparation (b) includes as the thickening polymer xanthan gum and/or the physiologically acceptable salt thereof and

and preparations (a) and (b) include no other thickening polymers apart from xanthan gum and/or the physiologically acceptable salt thereof.

Likewise suitable is also a product for the oxidative coloring and/or lightening of keratinic fibers, include

(1) a dispenser, which

has two chambers (A) and (B) separate from one another,

has an outlet opening (C), which is in communication with chamber (A) and with chamber (B),

(2) a preparation (a) in chamber (A), including in an aqueous, cosmetic carrier one or more thickening polymers in a total amount by weight (G1) of 0.1 to 40% by weight, based on the total weight of preparation (a),
(3) a preparation (b) in chamber (B), including in an aqueous cosmetic carrier

one or more thickening polymers in a total amount by weight (G2) of 0.1 to 40% by weight, based on the total weight of preparation (b),

characterized in that

the weight ratio G1/G2 has a value of 0.3 to 3.0, preferably of 0.5 to 2.0, more preferably of 0.55 to 1.8, even more preferably of 0.6 to 1.6, and particularly preferably in 0.7 to 1.4,

preparation (a) includes as the thickening polymer at least one acrylic acid polymer, an acrylic acid copolymer, a methacrylic acid polymer, and/or methacrylic acid polymers, and

preparation (b) includes as the thickening polymer at least one polysaccharide.

Preparations (a) and (b) can include as the thickening polymer also at least one acrylic acid polymer and/or at least one methacrylic acid polymer. An acrylic acid polymer is understood to mean a homopolymer of acrylic acid or a homopolymer of an acrylic acid derivative. A methacrylic acid polymer is understood to mean a homopolymer of methacrylic acid or a homopolymer of a methacrylic acid derivative.

Preferred acrylic acid and methacrylic acid polymers include as repeat units structures of the formula (P-I),

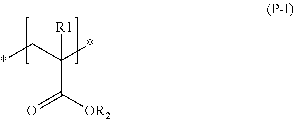

where
R1 stands for a hydrogen atom or a methyl group and
R2 stands for a hydrogen atom, for a C₁-C₃₀ alkyl group, a hydroxy-C₂-C₃₀-alkyl group, a C₁-C₃₀-alkoxy-C₂-C₆-alkyl group, for a C₁-C₃₀-alkyl-(CH₂—CH₂—O)ₙ—O-group, a C₁-C₆-dialkylamino-C₂-C₆-alkyl group, or for sodium, potassium, ½ magnesium, or ½ calcium,
n stands for a number from 1 to 30.

In the case of the preferred acrylic acid polymers, R1 stands for a hydrogen atom, and in the case of the preferred methacrylic acid polymers, R1 stands for a methyl group.

A preferred homopolymer of acrylic acid is a polyacrylic acid such as can be obtained, for example, from the company 3V Sigma under the trade names Synthalen K or Synthalen M or from the company Lubrizol under the trade names Carbopol (for example, Carbopol 980, 981, 954, 2984, and 5984), in each case with the INCI name Carbomer. The product sold by BASF under the trade name Cosmedia SP (INCI Name: SODIUM POLYACRYLATE) can be cited in this regard as a preferred acrylic acid homopolymer.

A further preferred embodiment is a product for the oxidative dyeing and/or lightening of keratinic fibers, include

(1) a dispenser, which

has two chambers (A) and (B) separate from one another,

has an outlet opening (C), which is in communication with chamber (A) and with chamber (B),

(2) a preparation (a) in chamber (A), including in an aqueous cosmetic carrier

one or more thickening polymers in a total amount by weight (G1) of 0.1 to 40% by weight, based on the total weight of preparation (a),

(3) a preparation (b) in chamber (B), including in an aqueous cosmetic carrier

one or more thickening polymers in a total amount by weight (G2) of 0.1 to 40% by weight, based on the total weight of preparation (b),

characterized in that

the weight ratio G1/G2 has a value of 0.3 to 3.0, preferably of 0.5 to 2.0, more preferably of 0.55 to 1.8, even more preferably of 0.6 to 1.6, and particularly preferably in 0.7 to 1.4,

preparation (a) includes as thickening polymer(s) at least one acrylic acid and/or methacrylic acid polymer with repeat units of the formula (P-I),

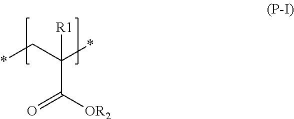

where
R1 stands for a hydrogen atom or a methyl group and
R2 stands for a hydrogen atom, for a C₁-C₃₀ alkyl group, a hydroxy-C₂-C₃₀-alkyl group, a C₁-C₃₀-alkoxy-C₂-C₆-alkyl group, for a C₁-C₃₀-alkyl-(CH₂—CH₂—O)ₙ—O-group, a C₁-C₆-dialkylamino-C₂-C₆-alkyl group, or for sodium, potassium, ½ magnesium, or ½ calcium,
n stands for a number from 1 to 30,

preparation (b) includes as thickening polymer(s) at least one acrylic acid and/or methacrylic acid polymer with repeat units of the formula (P-I*),

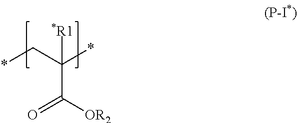

where
R1* stands for a hydrogen atom or a methyl group and
R2* stands for a hydrogen atom, for a C₁-C₃₀ alkyl group, a hydroxy-C₂-C₃₀-alkyl group, a C₁-C₃₀-alkoxy-C₂-C₆-alkyl group, for a C₁-C₃₀-alkyl-(CH₂—CH₂—O)ₙ—O-group, a C₁-C₆-dialkylamino-C₂-C₆-alkyl group, or for sodium, potassium, ½ magnesium, or ½ calcium,
n stands for a number from 1 to 30.

Furthermore, preparations (a) and (b) can include as the thickening polymer also at least one acrylic acid copolymer and/or a methacrylic acid copolymer.

Acrylic acid copolymers are understood to mean copolymers that are prepared by polymerization of acrylic acid or acrylic acid derivatives with one or more monomers different from acrylic acid or acrylic acid derivatives. Methacrylic acid copolymers are understood to mean copolymers that are prepared by polymerization of methacrylic acid or methacrylic acid derivatives with one or more monomers different from methacrylic acid or methacrylic acid derivatives.

Preferred acrylic acid and methacrylic acid copolymers include as repeat units structures of the formulas (P-II) and (P-III)

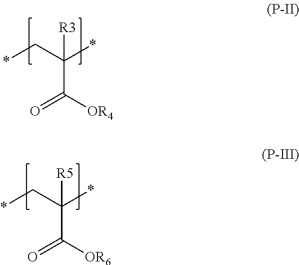

where
R3, R5 independently of one another stand for a hydrogen atom or a methyl group, R4 stands for a hydrogen atom, for a C₁-C₃₀ alkyl group, a hydroxy-C₂-C₃₀-alkyl group, a C₁-C₃₀-alkoxy-C₂-C₆-alkyl group, for a C₁-C₃₀-alkyl-(CH₂—CH₂—O)ₙ—O-group, a C₁-C₆-dialkylamino-C₂-C₆-alkyl group, or for sodium, potassium, ½ magnesium, or ½ calcium,
R6 stands for a hydrogen atom, for a C₁-C₃₀ alkyl group, a hydroxy-C₂-C₃₀-alkyl group, a C₁-C₃₀-alkoxy-C₂-C₆-alkyl group, for a C₁-C₃₀-alkyl-(CH₂—CH₂—O)ₘ—O-group, a C₁-C₆-dialkylamino-C₂-C₆-alkyl group, or for sodium, potassium, ½ magnesium, or ½ calcium,
n stands for a number from 1 to 30,
m stands for a number from 1 to 30.

Preferably, R3 and R5 are different from one another and/or R4 and R6 are different from another.

A further preferred embodiment is a product for the oxidative dyeing and/or lightening of keratinic fibers, include

(1) a dispenser, which

has two chambers (A) and (B) separate from one another,

has an outlet opening (C), which is in communication with chamber (A) and with chamber (B),

(2) a preparation (a) in chamber (A), including in an aqueous cosmetic carrier

one or more thickening polymers in a total amount by weight (G1) of 0.1 to 40% by weight, based on the total weight of preparation (a),

(3) a preparation (b) in chamber (B), including in an aqueous cosmetic carrier

one or more thickening polymers in a total amount by weight (G2) of 0.1 to 40% by weight, based on the total weight of preparation (b), characterized in that

the weight ratio G1/G2 has a value of 0.3 to 3.0, preferably of 0.5 to 2.0, more preferably of 0.55 to 1.8, even more preferably of 0.6 to 1.6, and particularly preferably in 0.7 to 1.4,

preparation (a) includes as thickening polymer(s) at least one acrylic acid copolymer and/or a methacrylic acid copolymer with repeat units of the formulas (P-II) and (P-III),

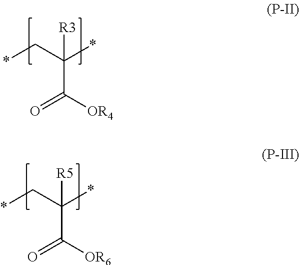

where
R3, R5 independently of one another stand for a hydrogen atom or a methyl group,
R4 stands for a hydrogen atom, for a C₁-C₃₀ alkyl group, a hydroxy-C₂-C₃₀-alkyl group, a C₁-C₃₀-alkoxy-C₂-C₆-alkyl group, for a C₁-C₃₀-alkyl-(CH₂—CH₂—O)ₙ—O-group, a C₁-C₆-dialkylamino-C₂-C₆-alkyl group, or for sodium, potassium, ½ magnesium, or ½ calcium,
R6 stands for a hydrogen atom, for a C₁-C₃₀ alkyl group, a hydroxy-C₂-C₃₀-alkyl group, a C₁-C₃₀-alkoxy-C₂-C₆-alkyl group, for a C₁-C₃₀-alkyl-(CH₂—CH₂—O)ₘ—O-group, a C₁-C₆-dialkylamino-C₂-C₆-alkyl group, or for sodium, potassium, ½ magnesium, or ½ calcium,
n stands for a number from 1 to 30,
m stands for a number from 1 to 30, and

preparation (b) includes as thickening polymer(s) at least one acrylic acid copolymer and/or a methacrylic acid copolymer with repeat units of the formulas (P-II*) and (P-III*),

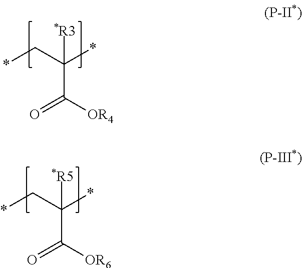

where
R3*, R5* independently of one another stand for a hydrogen atom or a methyl group,
R4* stands for a hydrogen atom, for a C₁-C₃₀ alkyl group, a hydroxy-C₂-C₃₀-alkyl group, a C₁-C₃₀-alkoxy-C₂-C₆-alkyl group, for a C₁-C₃₀-alkyl-(CH₂—CH₂—O)ₙ—O-group, a C₁-C₆-dialkylamino-C₂-C₆-alkyl group, or for sodium, potassium, ½ magnesium, or ½ calcium,
R6* stands for a hydrogen atom, for a C₁-C₃₀ alkyl group, a hydroxy-C₂-C₃₀-alkyl group, a C₁-C₃₀-alkoxy-C₂-C₆-alkyl group, for a C₁-C₃₀-alkyl-(CH₂—CH₂—O)ₘ—O-group, a C₁-C₆-dialkylamino-C₂-C₆-alkyl group, or for sodium, potassium, ½ magnesium, or ½ calcium,
n stands for a number from 1 to 30,
m stands for a number from 1 to 30.

Preferred acrylic acid and methacrylic acid copolymers are the sodium acrylate/sodium acryloyldimethyl taurate copolymers sold under the name Simulgel®EG as a compound with isohexadecane and polysorbate-80 (INCI-Name: SODIUM ACRYLATES/SODIUM ACRYLOYLDIMETHYL TAURATE COPOLYMER, ISOHEXADECANE, POLYSORBATE 80).

A polymer suitable in this regard, furthermore, is the polymer known under the INCI name Acrylates/C10-30 Alkyl Acrylates Crosspolymer, which can be obtained under the trade name Carbopol 1382 from the company Noveon. A polymer, suitable furthermore, is the polymer known under the INCI name Acrylates/Steareth-20 Methacrylate Crosspolymer, which is sold, for example, with the trade name Aculyn® 88 by the company Rohm & Haas in the form of a 28 to 30% by weight dispersion in water. Further, polymers known according to the INCI nomenclature as Acrylates/Palmeth-25 Acrylate Copolymer or Acrylates/Palmeth-20 Acrylate Copolymer can be used. Such polymers can be obtained, for example, under the trade name Synthalen® W 2000 as a 30 to 32% by weight emulsion in water from the company 3V Sigma.

It can likewise be preferable to use as the acrylic acid and/or methacrylic acid copolymer a copolymer of at least one anionic acrylic acid or methacrylic acid monomer and at least one nonionogenic monomer. Preferred nonionogenic monomers in this regard are acrylamide, methacrylamide, acrylic acid esters, methacrylic acid esters, vinylpyrrolidone, vinyl ethers, and vinyl esters.

Other preferred anionic copolymers are, for example, copolymers of acrylic acid and/or methacrylic acid, and C₁-C₆ alkyl esters thereof, as they are sold under the INCI declaration Acrylates Copolymers. A preferred commercial product is, for example, Aculyn® 33 of the company Rohm & Haas. Preferred further, however, are also copolymers of acrylic acid and/or methacrylic acid, C₁-C₆ alkyl esters of acrylic acid and/or methacrylic acid, as well as esters of an ethylenically unsaturated acid and an alkoxylated fatty alcohol. Suitable ethylenically unsaturated acids are in particular acrylic acid, methacrylic acid, and itaconic acid; suitable alkoxylated fatty alcohols are in particular Steareth-20 or Ceteth-20. Such copolymers are sold by the company Rohm & Haas under the trade name Aculyn® 22 (INCI Name: Acrylates/Steareth-20 Methacrylate Copolymer).

Other preferred acrylic acid or methacrylic acid copolymers are acrylic acid-acrylamide copolymers.

Preparations (a) and (b) can also include the following zwitterionic acrylic acid or methacrylic acid polymers:

copolymers of dimethyldiallylammonium salts and acrylic acid, e.g., Polyquaternium-22,

copolymers of dimethyldiallylammonium salts and methacrylic acid,

copolymers of N,N,N-trimethyl-3-[(2-methyl-1-oxo-2-propen-1-yl)amino]-1-propanaminium salts and acrylic acid,

copolymers of N,N,N-trimethyl-3-[(2-methyl-1-oxo-2-propen-1-yl)amino]-1-propanaminium salts and methacrylic acid,

copolymers of N,N,N-trimethyl-2-[(2-methyl-1-oxo-2-propen-1-yl)amino]-1-ethanaminium salts and acrylic acid,

copolymers of N,N,N-trimethyl-2-[(2-methyl-1-oxo-2-propen-1-yl)amino]-1-ethanaminium salts and methacrylic acid,

copolymers of N,N,N-trimethyl-3-[(2-methyl-1-oxo-2-propen-1-yl)amino]-1-propanaminium salts, acrylic acid, and acrylamide, e.g., Polyquaternium-53,

copolymers of N,N,N-trimethyl-3-[(2-methyl-1-oxo-2-propen-1-yl)amino]-1-propanaminium salts, methacrylic acid, and acrylamide,

copolymers of 1-ethenyl-3-methyl-1H-imidazolium salts, 1-ethenyl-1H-imidazole, 1-ethenyl-2-pyrrolidinone, and methacrylic acid, e.g., Polyquaternium-86,

copolymers of 1-ethenyl-3-methyl-1H-imidazolium salts, 1-ethenyl-1H-imidazole, 1-ethenyl-2-pyrrolidinone, and acrylic acid.

Preparations (a) and (b) of the invention can also include mixtures of the aforementioned preferred thickening polymers, whereby the requirement again applies that the ratio of the total amounts by weight of the thickening polymers (G1) and (G2) in preparations (a) and (b) has a value of 0.3 to 3.0.

Over the course of the work leading to this invention, it emerged that it is especially advantageous, if the thickening polymers in preparations (a) and (b) are each used in specific amount ranges.

In this case, the complete emptying of the dispenser via the outlet opening (C) could be assured particularly when the total amounts of weight (G1) and (G2) of the thickening polymers in preparations (a) and (b) were each in an amount range between 0.8 and 2.5% by weight. If the total amounts by weight (G1) and (G2) were selected too high (e.g., above 9.5% by weight), formation of clumps occurred within the dispenser or during the removal. If the total amounts by weight (G1) and (G2), in contrast, were selected too low, it was possible to match the rheological properties of both preparations (a) and (b) only insufficiently, so that the composition of the application mixture (of (a) and (b)) changes over the course of the application process (i.e., with the increasing emptying of the dispenser).

An especially preferred product of the invention is therefore characterized in that

preparation (a) includes the thickening polymer(s) in a total amount by weight (G1) of 0.2 to 9.5% by weight, preferably of 0.4 to 7.5% by weight, more preferably of 0.6 to 5.5% by weight, and particularly preferably of 0.8 to 2.5% by weight, based on the total weight of preparation (a), and

preparation (b) includes the thickening polymer(s) in a total amount by weight (G2) of 0.2 to 9.5% by weight, preferably of 0.4 to 7.5% by weight, more preferably of 0.6 to 5.5% by weight, and particularly preferably of 0.8 to 2.5% by weight, based on the total weight of preparation (b).

All aforementioned preferred and especially preferred quantitative data again apply with the proviso that the weight ratio G1/G2 is within the range according to the invention.

An especially preferred product of the invention is characterized in that

preparation (a) includes the thickening polymer(s) in a total amount by weight (G1) of 0.2 to 9.5% by weight, based on the total weight of preparation (a), and

preparation (b) includes the thickening polymer(s) in a total amount by weight (G2) of 0.2 to 9.5% by weight, based on the total weight of preparation (b).

An especially preferred product of the invention is characterized in that

preparation (a) includes the thickening polymer(s) in a total amount by weight (G1) of 0.4 to 7.5% by weight, based on the total weight of preparation (a), and

preparation (b) includes the thickening polymer(s) in a total amount by weight (G2) of 0.4 to 7.5% by weight, based on the total weight of preparation (b).

An especially preferred product of the invention is characterized in that

preparation (a) includes the thickening polymer(s) in a total amount by weight (G1) of 0.6 to 5.5% by weight, based on the total weight of preparation (a), and

preparation (b) includes the thickening polymer(s) in a total amount by weight (G2) of 0.6 to 5.5% by weight, based on the total weight of preparation (b).

An especially preferred product of the invention is characterized in that

preparation (a) includes the thickening polymer(s) in a total amount by weight (G1) of 0.8 to 2.5% by weight, based on the total weight of preparation (a), and

preparation (b) includes the thickening polymer(s) in a total amount by weight (G2) of 0.8 to 2.5% by weight, based on the total weight of preparation (b).

The use of similar thickening polymers in mutually matched amounts by weight in preparations (a) and (b) assures the removal of constant mass ratios of both formulations via outlet opening (C), because the rheological properties of both formulations can be reliably matched to one another in this way. The use of the special thickeners assures hereby primarily a similar flow behavior in preparations (a) and (b), which otherwise have a very different composition, a behavior that is also little affected by differences in dispenser geometry. In this case, the maintenance of special viscosity ranges has proven to be very especially advantageous.

Very especially preferred therefore is a product for the oxidative color changing of keratinic fibers, which is characterized in that

preparation (a) has a viscosity of 1000 to 100,000 mPas, preferably of 2000 to 80,000 mPas, more preferably of 4000 to 60,000 mPas, even more preferably of 7000 to 50,000 mPas, and particularly preferably of 10,000 to 40,000 mPas (22° C./Brookfield viscometer/spindle 5/4 rpm),

preparation (b) has a viscosity of 1000 to 100,000 mPas, preferably of 2000 to 80,000 mPas, more preferably of 4000 to 60,000 mPas, even more preferably of 7000 to 50,000 mPas, and particularly preferably of 10,000 to 40,000 mPas (22° C./Brookfield viscometer/spindle 5/4 rpm), and

the ratio of the viscosities V1/V2 has a value of 0.3 to 3.0, preferably of 0.4 to 2.5, and particularly preferably of 0.5 to 2.0.

The indicated viscosities are viscosities that were measured at 22° C. (22° C./Brookfield viscometer/spindle 5/4 rpm).

The rheological properties of preparations (a) and (b) can be matched even better to one another, if both preparations in addition have a similar or the same salt content. In another especially preferred embodiment, preparations (a) and (b) are therefore characterized in addition by their mutually matched salt content. Preparation (a) preferably includes one or more organic and/or inorganic salts in a total molality (M1) of 0.01 to 1 mol/kg, based on the total weight of preparation (a). Preparation (b) includes further one or more organic and/or inorganic salts in a total molality (M2) of 0.01 to 1 mol/kg, based on the total weight of preparation (b). It is essential to the invention in this case that the molality ratio M1/M2 has a value of 0.3 to 3.0.

In another very especially preferred embodiment, a product of the invention is characterized in that

preparation (a) includes in addition one or more organic and/or inorganic salts in a total molality (M1) of 0.01 to 1 mol/kg, based on the total weight of preparation (a),

preparation (b) includes in addition one or more organic and/or inorganic salts in a total molality (M2) of 0.01 to 1 mol/kg, based on the total weight of preparation (b), and

the molality ratio M1/M2 has a value of 0.3 to 3.0.

Organic and/or inorganic salts are understood to mean all charged, i.e., ionic compounds of a nonpolymeric nature. The compounds are classified as nonpolymeric when they have a molar weight of a maximum of 5000 g/mol.

Polymeric compounds (i.e., compounds with a molar mass of more than 5000 g/mol) are not included in the definition of organic salts, even if they have cationic and/or anionic charges. The reason for this is that they influence the rheological properties of preparation (a) and (b) not only based on their charges, but particularly based on their polymeric structure (particularly on their thickening properties). Charged polymeric compounds are therefore not considered in calculating the total molalities M1 and M2.

Examples of inorganic salts are alkali metal sulfates, alkali metal chlorides, alkali metal bromides, alkali metal sulfates, alkali metal hydrogen sulfates, alkali metal phosphates, alkali metal hydroxides, alkali metal phosphates, alkali metal hydrogen phosphates, alkaline earth metal sulfites, alkaline earth metal chlorides, alkaline earth metal bromides, alkaline earth metal sulfates, alkaline earth metal hydrogen sulfates, alkaline earth metal phosphates, alkaline earth metal hydroxides, alkaline earth metal phosphates, and alkaline earth metal hydrogen phosphates.

Examples of organic salts are charged surfactants, i.e., cationic surfactants, anionic surfactants, and also zwitterionic surfactants, in the form of their inner salt.

Other examples of organic salts are the oxidation dye precursors present in the form of their salts. Oxidation dye precursors can be divided into developers and couplers, whereby the developers because of their rather high sensitivity to oxygen are used mostly in the form of their physiologically acceptable salts (e.g., in form of their hydrochlorides, hydrobromides, hydrogen sulfates, or sulfates).

Coupler components during oxidative coloring alone cause no significant coloring, but always require the presence of developer components. Because couplers are not as sensitive to oxygen as developers, they can in fact also be used in the form of their salts in the preparations, but are often also used in the free form (i.e., not in the salt form). All developers and couplers, which are used in the form of their salt in the formulation, fall within the group of organic salts.

Preferred salts of the developer type are p-phenylenediamine×H₂SO₄, p-phenylenediamine×2 HCl, p-toluylenediamine×H₂SO₄, p-toluylenediamine×2 HCl, 2-(2-hydroxyethyl)-p-phenylenediamine×H₂SO₄, 2-(2-hydroxyethyl)-p-phenylenediamine×2 HCl, 2-(1,2-dihydroxyethyl)-p-phenylenediamine×H₂SO₄, 2-(1,2-dihydroxyethyl)-p-phenylenediamine×2 HCl, N,N-bis(2-hydroxyethyl)-p-phenylenediamine×H₂SO₄, N,N-bis(2-hydroxyethyl)-p-phenylenediamine×2 HCl, 2-methoxymethyl-p-phenylenediamine×H₂SO₄, 2-methoxymethyl-p-phenylenediamine×2 HCl, N-(4-amino-3-methylphenyl)-N-[3-(1H-imidazol-1-yl)propyl]amine×H₂SO₄, N-(4-amino-3-methylphenyl)-N-[3-(1H-imidazol-1-yl)propyl]amine×2 HCl, N-(4-Amino-3-methylphenyl)-N-[3-(1H-imidazol-1-yl)propyl]amine×3 HCl, bis(2-hydroxy-5-aminophenyl)methane×H₂SO₄, bis(2-hydroxy-5-aminophenyl)methane×2 HCl, 4,5-diamino-1-(2-hydroxyethyl)-1H-pyrazole×H₂SO₄, 4,5-diamino-1-(2-hydroxyethyl)-1H-pyrazole×2 HCl, 2,4,5,6-tetraaminopyrimidine×H₂SO₄, 2,4,5,6-tetraaminopyrimidine×2H₂SO₄, 2,4,5,6-tetraaminopyrimidine×2 HCl, 2,4,5,6-tetraaminopyrimidine×3 HCl, 2,4,5,6-tetraaminopyrimidine×4 HCl, 4-hydroxy-2,5,6-triaminopyrimidine×H₂SO₄, 4-hydroxy-2,5,6-triaminopyrimidine×2 HCl, 2-hydroxy-4,5,6-triaminopyrimidine×H₂SO₄, 2-hydroxy-4,5,6-triaminopyrimidine×2 HCl, and 2-hydroxy-4,5,6-triaminopyrimidine×3 HCl.

Total molality (M1) is understood to mean the total molar amount of all organic and inorganic salts included in preparation (a), whereby M1 is based on the total weight of preparation (a) (unit: mole of salt per kg of formulation (a)). All organic and inorganic salts that are added to preparation (a) in the form of their salts are included in calculating the total molality M1.

Total molality (M2) is understood to mean the total molar amount of all organic and inorganic salts included in preparation (b), whereby M2 is based on the total weight of preparation (b) (unit: mole of salt per kg of formulation (b)). All organic and inorganic salts that are added to preparation (b) in the form of their salts are included in calculating the total molality M2.

The ratio of the molalities M1/M2 is a ratio of 0.3 to 3.0. In this case, the weight ratio of formulations (a) and (b), which is removed from the dispenser during use, is the more constant and defined, the more similar the salt molalities used in both formulations, i.e., the closer the molality ratio M1/M2 is to a value of 1.

In another very especially preferred embodiment, a product for the oxidative coloring and/or lightening of keratinic fibers is characterized therefore in that the molality ratio M1/M2 has a value of 0.4 to 2.5, preferably of 0.5 to 2.0, more preferably of 0.6 to 1.7, and particularly preferably of 0.7 to 1.4.

Furthermore, very especially preferred for this reason is a product for the oxidative coloring and/or lightening of keratinic fibers, include

(1) a dispenser, which

has two chambers (A) and (B) separate from one another,

has an outlet opening (C), which is in communication with chamber (A) and with chamber (B),

(2) a preparation (a) in chamber (A), including in an aqueous cosmetic carrier

one or more thickening polymers in a total amount by weight (G1) of 0.1 to 40% by weight, based on the total weight of preparation (a),

one or more organic and/or inorganic salts in a total molality (M1) of 0.01 to 1 mol/kg, based on the total weight of preparation (a),

(3) a preparation (b) in chamber (B), including in an aqueous cosmetic carrier

one or more thickening polymers in a total amount by weight (G2) of 0.1 to 40% by weight, based on the total weight of preparation (b),

one or more organic and/or inorganic salts in a total molality (M2) of 0.01 to 1 mol/kg, based on the total weight of preparation (b),

characterized in that

the weight ratio G1/G2 has a value of 0.5 to 2.0 and

the molality ratio M1/M2 has a value of 0.3 to 3.0, preferably of 0.4 to 2.5, more preferably of 0.5 to 2.0, even more preferably of 0.6 to 1.7, and preferably of 0.7 to 1.4.

Furthermore, very especially preferred for this reason is a product for the oxidative coloring and/or lightening of keratinic fibers, include

(1) a dispenser, which

has two chambers (A) and (B) separate from one another,

has an outlet opening (C), which is in communication with chamber (A) and with chamber (B),

(2) a preparation (a) in chamber (A), including in an aqueous cosmetic carrier

one or more thickening polymers in a total amount by weight (G1) of 0.1 to 40% by weight, based on the total weight of preparation (a),

one or more organic and/or inorganic salts in a total molality (M1) of 0.01 to 1 mol/kg, based on the total weight of preparation (a),

(3) a preparation (b) in chamber (B), including in an aqueous cosmetic carrier

one or more thickening polymers in a total amount by weight (G2) of 0.1 to 40% by weight, based on the total weight of preparation (b),

one or more organic and/or inorganic salts in a total molality (M2) of 0.01 to 1 mol/kg, based on the total weight of preparation (b),

characterized in that

the weight ratio G1/G2 has a value of 0.55 to 1.8 and

the molality ratio M1/M2 has a value of 0.3 to 3.0, preferably of 0.4 to 2.5, more preferably of 0.5 to 2.0, even more preferably of 0.6 to 1.7, and preferably of 0.7 to 1.4.

Furthermore, very especially preferred for this reason is a product for the oxidative coloring and/or lightening of keratinic fibers, include

(1) a dispenser, which

has two chambers (A) and (B) separate from one another,

has an outlet opening (C), which is in communication with chamber (A) and with chamber (B),

(2) a preparation (a) in chamber (A), including in an aqueous cosmetic carrier

one or more thickening polymers in a total amount by weight (G1) of 0.1 to 40% by weight, based on the total weight of preparation (a),

one or more organic and/or inorganic salts in a total molality (M1) of 0.01 to 1 mol/kg, based on the total weight of preparation (a),

(3) a preparation (b) in chamber (B), including in an aqueous cosmetic carrier

one or more thickening polymers in a total amount by weight (G2) of 0.1 to 40% by weight, based on the total weight of preparation (b),

one or more organic and/or inorganic salts in a total molality (M2) of 0.01 to 1 mol/kg, based on the total weight of preparation (b),

characterized in that

the weight ratio G1/G2 has a value of 0.6 to 1.6 and

the molality ratio M1/M2 has a value of 0.3 to 3.0, preferably of 0.4 to 2.5, more preferably of 0.5 to 2.0, even more preferably of 0.6 to 1.7, and preferably of 0.7 to 1.4.

Furthermore, especially preferred for this reason is a product for the oxidative coloring and/or lightening of keratinic fibers, include (1) a dispenser, which

has two chambers (A) and (B) separate from one another,

has an outlet opening (C), which is in communication with chamber (A) and with chamber (B),

(2) a preparation (a) in chamber (A), including in an aqueous cosmetic carrier

one or more thickening polymers in a total amount by weight (G1) of 0.1 to 40% by weight, based on the total weight of preparation (a),

one or more organic and/or inorganic salts in a total molality (M1) of 0.01 to 1 mol/kg, based on the total weight of preparation (a),

(3) a preparation (b) in chamber (B), including in an aqueous cosmetic carrier

one or more thickening polymers in a total amount by weight (G2) of 0.1 to 40% by weight, based on the total weight of preparation (b),

one or more organic and/or inorganic salts in a total molality (M2) of 0.01 to 1 mol/kg, based on the total weight of preparation (b),

characterized in that

the weight ratio G1/G2 has a value of 0.7 to 1.4 and

the molality ratio M1/M2 has a value of 0.3 to 3.0, preferably of 0.4 to 2.5, more preferably of 0.5 to 2.0, even more preferably of 0.6 to 1.7, and preferably of 0.7 to 1.4.

Because of the different functions of preparations (a) and (b), both preparations include by definition different salts, however.

An especially preferred product is characterized in that preparation (a) includes one or more organic and/or inorganic salts from the group of salt-like oxidation dye precursors, alkali metal salts of 1-hydroxyethane-1,1-diphosphonic acid, alkali metal sulfites, alkali metal chlorides, alkali metal bromides, alkaline earth metal chlorides, alkaline earth metal bromides, alkali metal sulfates, alkaline earth metal sulfates, alkali metal hydrogen sulfates, alkaline earth metal hydrogen sulfates, alkali metal phosphates, and/or alkaline earth metal phosphates.

In another especially preferred embodiment, a product for the oxidative color change is characterized in that preparation (b) include one or more organic and/or inorganic salts from the group of the alkali metal salts of 1-hydroxyethane-1,1-diphosphonic acid, the alkali metal salts of dipicolinic acid, alkali metal hydroxides, alkali metal benzoates, alkali metal chlorides, alkaline earth metal chlorides, alkali metal bromides, alkaline earth metal bromides, alkali metal carbonates, alkaline earth metal hydrogen carbonates, alkali metal sulfates, alkaline earth metal sulfates, alkali metal hydrogen sulfates, alkaline earth metal hydrogen sulfates, alkali metal phosphates, alkaline earth metal phosphates, alkali metal pyrophosphates, and/or alkaline earth metal pyrophosphates.

The total molalities of the salts included in preparations (a) and (b) are hereby matched to one another, however, as previously described, which means that molality ratio M1/M2 is in the value range according to the invention or in the preferred or especially preferred value range. The best dispensability is achieved with the most similar salt contents possible.

Used in formulations (a) and (b) hereby can be both salts of singly positively charged cations with singly negatively charged anions (e.g., sodium chloride), and also the salts of multiply positively charged cations with singly negatively charged anions (e.g., magnesium chloride), as well as the salts of singly positively charged cations with multiply negatively charged anions (e.g., sodium sulfate), and finally also the salts of multiply positively charged cations with multiply negatively charged anions (e.g., magnesium sulfate), in each case with the maintenance of the stoichiometric conditions necessary for the total neutrality of the salt.

It could be observed in this regard that not only the concentration (i.e., molality) of the salts included in preparations (a) and (b) exerts an effect on the dispensability of preparations (a) and (b), but that moreover salts with an ionic charge as similar as possible as well should be used in both formulations (a) and (b).

In other words, application mixtures with a reproducible composition can be removed from the product especially when formulations (a) and (b) included organic/inorganic salts not only in similar amounts but when the ionic charge as well of the salts employed in (a) and (b) was as similar as possible. The ionic charge of the employed salts can be quantified via the ionic strength. The ionic strength is a charge-dependent concentration measure for electrolytic solutions.

In another especially preferred embodiment, a product for the oxidative color changing of keratinic fibers is characterized in that

preparation (a) includes one or more organic and/or inorganic salts in an ionic strength (I1), calculated according to the formula (1)

$$I 1 = \frac{1}{2} {\sum }_{i} {z}_{i}^{2} ci \qquad ( 1 )$$

where zi is the ionic charge of each ion (i) in preparation (a) and
ci is the molality (mol/kg) of each ion in preparation (a), whereby the molality is based on the total weight of preparation (a),

preparation (b) includes one or more organic and/or inorganic salts in an ionic strength (I2), calculated according to the formula (2)

$$I 2 = \frac{1}{2} {\sum }_{i} {z}_{i}^{2} ci \qquad ( 2 )$$

where zi is the ionic charge of each ion (i) in preparation (b) and
ci is the molality (mol/kg) of each ion in preparation (b), whereby the molality is based on the total weight of preparation (b), and

the ratio of the ionic strengths I1/I2 has a value of 0.5 to 2.0, preferably of 0.6 to 1.8, and more preferably of 0.7 to 1.6.

The ionic strengths I1 and I2 according to the above definition take into account the charges and molalities of each individual ion in preparations (a) and (b).

The calculation basis for the molality ci of the ionic strength I1 is the total weight of preparation (a). The calculation basis for the molality ci of the ionic strength I2 is the total weight of preparation (b).

### Example

Preparation (a) includes 0.2% by weight of p-toluylenediamine, sulfate salt (C₇H₁₂N₂²⁺×SO₄²⁻) (molar mass=220.25 g/mol);

1000 g of formulation (a) includes 2 g of p-toluylenediamine, sulfate salt=9.08×10⁻³ mol Molality=9.08×10⁻³ mol/kg, ionic charge cation=2, ionic charge anion=2
I1=2×(9.08×10⁻³) mol/kg (C₇H₁₂N₂²⁺)+2×(9.08×10⁻³) mol/kg (SO₄²⁻)=0.03632

Preparation (b) includes 0.11% by weight of magnesium sulfate (MgSO₄) (molar mass=120.37 g/mol)

1000 g of formulation (b) includes 1.1 g of magnesium sulfate=9.14×10⁻³ mol
Molality=9.14×10⁻³ mol/kg, ionic charge cation=2, ionic charge anion=2
I2=2×(9.14×10⁻³ mol/kg)+2×9.14×10⁻³ mol/kg=0.03655 I1/I2=0.994

Preparation (a) can include in addition also one or more oxidation dye precursors, preferably at least one oxidation dye precursor of the developer type and at least one oxidation dye precursor of the coupler type. Especially suitable oxidation dye precursors of the developer type are thereby selected from at least one compound from the group, which is formed by p-phenylenediamine, p-toluylenediamine, 2-(2-hydroxyethyl)-p-phenylenediamine, dihydroxyethyl)-p-phenylenediamine, N,N-bis(2-hydroxyethyl)-p-phenylenediamine, 2-methoxymethyl-p-phenylenediamine, N-(4-amino-3-methylphenyl)-N-[3-(1H-imidazol-1-yl)-propyl]amine, N,N′-bis(2-hydroxyethyl)-N,N′-bis(4-aminophenyl)-1,3-diaminopropan-2-ol, bis(2-hydroxy-5-aminophenyl)methane, 1,3-bis(2,5-diaminophenoxy)propan-2-ol, N,N′-bis(4-aminophenyl)-1,4-diazacycloheptane, 1,10-bis(2,5-diaminophenyl)-1,4,7,10-tetraoxadecane, p-aminophenol, 4-amino-3-methylphenol, 4-amino-2-aminomethylphenol, 4-amino-2-(1,2-dihydroxyethyl)phenol, 4-amino-2-(diethylaminomethyl)phenol, 4,5-diamino-1-(2-hydroxyethyl)pyrazole, 2,4,5,6-tetraaminopyrimidine, 4-hydroxy-2, 5,6-triaminopyrimidine, 2-hydroxy-4,5,6-triaminopyrimidine, 2,3-diamino-6,7-dihydro-1H,5H-pyrazolo[1,2-a]pyrazol-1-one, and the physiologically acceptable salts thereof.

Especially suitable oxidation dye precursors of the coupler type in this case are selected from the group, formed by 3-aminophenol, 5-amino-2-methylphenol, 3-amino-2-chloro-6-methylphenol, 2-hydroxy-4-aminophenoxyethanol, 5-amino-4-chloro-2-methylphenol, 5-(2-hydroxyethyl)amino-2-methylphenol, 2,4-dichloro-3-aminophenol, 2-aminophenol, 3-phenylenediamine, 2-(2,4-diaminophenoxy)ethanol, 1,3-bis(2,4-diaminophenoxy)propane, 1-methoxy-2-amino-4-(2-hydroxyethylamino)benzene, 1,3-bis(2,4-diaminophenyl)propane, 2,6-bis(2′-hydroxyethylamino)-1-methylbenzene, 2-({3-[(2-hydroxyethyl)amino]-4-methoxy-5-methylphenyl}amino)ethanol, 2-({3-[(2-hydroxyethyl)amino]-2-methoxy-5-methylphenyl}amino)ethanol, 2-({3-[(2-hydroxyethyl)amino]-4,5-dimethylphenyl}amino)ethanol, 2-[3-morpholin-4-ylphenyl)amino]ethanol, 3-amino-4-(2-methoxyethoxy)-5-methylphenyl amine, 1-amino-3-bis(2-hydroxyethyl)aminobenzene, resorcinol, 2-methylresorcinol, 4-chlororesorcinol, 1,2,4-trihydroxybenzene, 2-amino-3-hydroxypyridine, 3-amino-2-methylamino-6-methoxypyridine, 2,6-dihydroxy-3,4-dimethylpyridine, 3,5-diamino-2,6-dimethoxypyridine, 1-phenyl-3-methylpyrazol-5-one, 1-naphthol, 1,5-dihydroxynaphthalene, 2,7-dihydroxynaphthalene, 1,7-dihydroxynaphthalene, 1,8-dihydroxynaphthalene, 4-hydroxyindole, 6-hydroxyindole, 7-hydroxyindole, 4-hydroxyindoline, 6-hydroxyindoline, 7-hydroxyindoline, or mixtures of said compounds or the physiologically acceptable salts thereof.

All oxidation dye precursors, used in their salt form, must be considered hereby in the calculation of the total molality M1.

In addition, preparation (a) can also include at least one direct dye from the group of anionic, nonionic, and/or cationic dyes. The anionic and cationic direct dyes fall in the group of organic salts and must also be considered in the calculation of the total molality M1.

The oxidation dye precursors, i.e., developer components and coupler components, and the optional additional direct dyes can be used, for example, in an amount of 0.0001 to 5.0% by weight, preferably 0.001 to 3.5% by weight, in each case based on the total weight of preparation (a).

A surface-active substance is preferably added to preparations (a) and (b), whereby such surface-active substances are called surfactants or emulsifiers depending on the field of application: they are preferably selected from anionic, cationic, zwitterionic, amphoteric, and nonionic surfactants and emulsifiers.

Preparations (a) and (b) preferred according to the invention are characterized in that they include in addition at least one anionic surfactant. Preferred anionic surfactants are fatty acids, alkyl sulfates, alkyl ether sulfates, and ether carboxylic acids having 10 to 20 C atoms in the alkyl group and up to 16 glycol ether groups in the molecule. The anionic surfactants are used in amounts of 0.1 to 45% by weight, preferably 1 to 30% by weight, and very especially preferably of 1 to 15% by weight, based on the total amount of the ready-to-use agent.

Preparations (a) and (b) preferred according to the invention are characterized in that preparations (a) and/or (b) include in addition at least one zwitterionic surfactant. Preferred zwitterionic surfactants are betaines, N-alkyl-N,N-dimethylammonium glycinates, N-acylaminopropyl-N,N-dimethylammonium glycinates, and 2-alkyl-3-carboxymethyl-3-hydroxyethylimidazolines. A preferred zwitterionic surfactant is known by the INCI name Cocamidopropyl Betaine.

Preparations (a) and (b) preferred according to the invention are characterized in that preparations (a) and/or (b) include in addition at least one amphoteric surfactant. Preferred amphoteric surfactants are N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids, and alkylaminoacetic acids. Especially preferred amphoteric surfactants are N-cocoalkyl aminopropionate, cocoacylaminoethyl aminopropionate, and C₁₂-C₁₈ acylsarcosine.

The nonionic, zwitterionic, or amphoteric surfactants can be used in proportions of 0.1 to 40% by weight, preferably 1 to 30% by weight, and very particularly preferably of 1 to 15% by weight, based in each case on the total amount of preparations (a) and (b).

All anionic, cationic, and zwitterionic surfactants included in preparations (a) and (b) fall in the group of organic salts, and their employed concentrations must therefore be considered in the calculation of the total molalities M1 or M2.

It has proven advantageous furthermore for the agents to include further non-ionogenic surface-active substances. Preferred nonionic surfactants are, for example, alkylene oxide addition products to fatty alcohols and fatty acids with in each case 2 to 30 mol of ethylene oxide per mole of fatty alcohol or fatty acid. Preparations with excellent properties are likewise obtained if they include fatty acid esters of ethoxylated glycerol as the nonionic surfactants.

In another totally especially preferred embodiment, preparations (a) and (b) also include in each case similar or the same amounts of nonionic surfactants.

In addition, preparations (a) and (b) can also include one or more fatty components. “Fatty components” within the meaning of the invention are understood to mean organic compounds with a solubility in water at room temperature (22° C.) and atmospheric pressure (760 mm Hg) of less than 1% by weight, preferably of less than 0.1% by weight.

The definition of fatty components includes explicitly only uncharged (i.e., nonionic) compounds. Fatty components have at least one saturated or unsaturated alkyl group having at least 8 C atoms. The molar weight of the fatty components is a maximum of 5000 g/mol, preferably a maximum of 2500 g/mol, and particularly preferably a maximum of 1000 g/mol. The fatty components are neither polyoxyalkylated nor polyglycerylated compounds.

Suitable fatty components are, for example, C₁₂-C₃₀ fatty alcohols, C₁₂-C₃₀ fatty acid triglycerides, C₁₂-C₃₀ fatty acid monoglycerides, C₁₂-C₃₀ fatty acid diglycerides, and/or hydrocarbons. Only nonionic substances are regarded explicitly as fatty components within the meaning of the present invention. Charged compounds such as, for example, fatty acids and salts thereof are not understood to be a fatty component.

C₁₂-C₃₀ fatty alcohols can be saturated, mono- or polyunsaturated, linear or branched fatty alcohols having 12 to 30 C atoms.

Examples of suitable linear, saturated C₁₂-C₃₀ fatty alcohols are dodecan-1-ol (dodecyl alcohol, lauryl alcohol), tetradecan-1-ol (tetradecyl alcohol, myristyl alcohol), hexadecan-1-ol (hexadecyl alcohol, cetyl alcohol, palmityl alcohol), octadecan-1-ol (octadecyl alcohol, stearyl alcohol), arachidyl alcohol (eicosan-1-ol), heneicosyl alcohol (heneicosan-1-ol), and/or behenyl alcohol (docosan-1-ol).

Suitable linear, unsaturated fatty alcohols are (9Z)-octadec-9-en-1-ol (oleyl alcohol), (9E)-octadec-9-en-1-ol (elaidyl alcohol), (9Z,12Z)-octadeca-9,12-dien-1-ol (linoleyl alcohol), (9Z,12Z,15Z)-octadeca-9,12,15-trien-1-ol (linolenoyl alcohol), gadoleyl alcohol ((9Z)-eicos-9-en-1-ol), arachidonyl alcohol ((5Z,8Z,11Z,14Z)-eicosa-5,8,11,14-tetraen-1-ol), erucyl alcohol ((13Z)-docos-13-en-1-ol), and/or brassidyl alcohol ((13E)-docosen-1-ol).

A C₁₂-C₃₀ fatty acid triglyceride within the meaning of the present invention is understood to mean the triesters of the trihydric alcohol, glycerol, with three equivalents of fatty acids. In this regard, both structurally similar and also different fatty acids can be involved in ester formations within a triglyceride molecule.

Fatty acids according to the invention are understood to mean saturated or unsaturated, unbranched or branched, unsubstituted or substituted C₁₂-C₃₀ carboxylic acids. Unsaturated fatty acids can be mono- or polyunsaturated. In the case of an unsaturated fatty acid, the C—C double bond(s) thereof can have the cis or trans configuration.

Cited by way of example for fatty acid mono-, di-, and triglycerides can be triglycerides in which at least one of the ester groups is formed originating from glycerol with a fatty acid, which is selected from dodecanoic acid (lauric acid), tetradecanoic acid (myristic acid), hexadecanoic acid (palmitic acid), tetracosanoic acid (lignoceric acid), octadecanoic acid (stearic acid), eicosanoic acid (arachidic acid), docosanoic acid (behenic acid), petroselinic acid [(Z)-6-octadecenoic acid], palmitoleic acid [(9Z)-hexadec-9-enoic acid], oleic acid [(9Z)-octadec-9-enoic acid], elaidic acid [(9E)-octadec-9-enoic acid], erucic acid [(13Z)-docos-13-enoic acid], linoleic acid [(9Z,12Z)-octadeca-9,12-dienoic acid], linolenic acid [(9Z,12Z,15Z)-octadeca-9,12,15-trienoic acid], eleostearic acid [(9Z,11E,13E)-octadeca-9,11,3-trienoic acid], arachidonic acid [(5Z,8Z,11Z,14Z)-eicosa-5,8,11,14-tetraenoic acid], and/or nervonic acid [(15Z)-tetracos-15-enoic acid].

The fatty acid triglycerides can also be of natural origin. The fatty acid triglycerides, occurring in soybean oil, peanut oil, olive oil, sunflower oil, macadamia nut oil, Moringa oil, apricot kernel oil, Marula oil, and/or optionally hydrogenated castor oil, or mixtures thereof are especially suitable for use in the product of the invention.

A C₁₂-C₃₀ fatty acid monoglyceride is understood to mean the monoester of the trihydric alcohol glycerol with a fatty acid equivalent. In this case, either the middle hydroxy group of glycerol or the terminal hydroxy group of glycerol can be esterified with the fatty acid.

A C₁₂-C₃₀ fatty acid diglyceride is understood to mean the diester of the trihydric alcohol, glycerol, with two fatty acid equivalents. In this case, either the middle and a terminal hydroxy group of glycerol can be esterified with two fatty acid equivalents, or however both terminal hydroxy groups of glycerol are esterified with one fatty acid in each case. Glycerol can be esterified hereby both with two structurally similar and with two different fatty acids.

Hydrocarbons are compounds consisting exclusively of carbon and hydrogen atoms and having 8 to 80 C atoms. Preferred in this regard are particularly aliphatic hydrocarbons such as, for example, mineral oils, liquid paraffin oils (e.g., liquid paraffin or light liquid paraffin), isoparaffin oils, semisolid paraffin oils, paraffin waxes, hard paraffin (solid paraffin), Vaseline, and polydecene.

Liquid paraffin oils (liquid paraffin and light liquid paraffin) have proven to be suitable in this regard. The hydrocarbon can be, for example, liquid paraffin, also called white oil. Liquid paraffin is a mixture of purified, saturated, aliphatic hydrocarbons, which consists for the most part of hydrocarbon chains with a C-chain distribution of 25 to 35 C atoms.

Even though preparations (a) and (b) can include fatty components, thus in an especially preferred embodiment they are yet free of fatty components.

The term “free of fatty components” within the meaning of the present invention is understood to mean that preparation (a) includes a total amount by weight of fatty components of less than 2.5% by weight, preferably of less than 1.0% by weight, and especially preferably less than 0.5% by weight, based on the total weight of preparation (a).

The term “free of fatty components” within the meaning of the present invention is understood to mean that preparation (b) includes a total amount by weight of fatty components of less than 2.5% by weight, preferably of less than 1.0% by weight, and especially preferably less than 0.5% by weight, based on the total weight of preparation (b).

Especially preferred, furthermore, is a product for the oxidative coloring and/or lightening of keratinic fibers, include

(1) a dispenser, which

has two chambers (A) and (B) separate from one another,

has an outlet opening (C), which is in communication with chamber (A) and with chamber (B),

(2) a preparation (a) in chamber (A), including in an aqueous cosmetic carrier

one or more thickening polymers in a total amount by weight (G1) of 0.1 to 40% by weight, based on the total weight of preparation (a),

(3) a preparation (b) in chamber (B), including in an aqueous cosmetic carrier

one or more thickening polymers in a total amount by weight (G2) of 0.1 to 40% by weight, based on the total weight of preparation (b),

characterized in that

the weight ratio G1/G2 has a value of 0.3 to 3.0 and

the total amount of fatty components in preparation (a) is less than 2.5% by weight, preferably less than 1.0% by weight, especially preferably less than 0.5% by weight, based on the total weight of preparation (a), and

the total amount of fatty components in preparation (b) is less than 2.5% by weight, preferably less than 1.0% by weight, especially preferably less than 0.5% by weight, based on the total weight of preparation (b).

Preparation (a) is preferably a formulation that is made alkaline and optionally includes oxidation dye precursors, if a color change is also desired in addition to the lightening. Preparation (b) is a formulation with oxidizing agents that is made acidic for reasons of stability.

In a further especially preferred embodiment, a product of the invention is therefore characterized in that

preparation (a) includes at least one alkalinizing agent, preferably ammonia and/or monoethanolamine, and

preparation (b) includes at least one oxidizing agent, preferably hydrogen peroxide.

Preparation (a) preferably includes at least one alkalinizing agent, preferably ammonia and/or monoethanolamine. Alkalinizing agents that can be used according to the invention can be selected from the group that is formed by ammonia, alkanolamines, basic amino acids, and inorganic alkalinizing agents such as alkali (alkaline earth) metal hydroxides, alkali (alkaline earth) metal metasilicates, alkali (alkaline earth) metal phosphates, and alkali (alkaline earth) metal hydrogen phosphates. Suitable inorganic alkalinizing agents are sodium hydroxide, potassium hydroxide, sodium silicate, and sodium metasilicate. Organic alkalinizing agents that can be used according to the invention can be selected from monoethanolamine, 2-amino-2-methylpropanol, and triethanolamine. The basic amino acids that can be used as alkalinizing agents according to the invention are preferably selected from the group formed by arginine, lysine, ornithine, and histidine, especially preferably arginine.

Ammonia (NH₃) is customarily employed in the form of its aqueous solution. Aqueous ammonia solutions include ammonia (NH₃) often in concentrations between 10% by weight and 32% by weight. Preferred in this case is the use of an aqueous ammonia solution, which includes 25% by weight of ammonia (NH₃).

Preferably, preparation (a) includes ammonia and/or monoethanolamine in amounts of 0.01 to 10% by weight, preferably of 0.1 to 7.5% by weight, more preferably of 0.2 to 5.5% by weight, and particularly preferably of 0.4 to 4.5% by weight, in each case based on the total weight of preparation (a).

If the alkalinizing agents are used in the salt form such as, for example, potassium hydroxide or sodium hydroxide, thus their employed concentrations must also be considered in calculating the total molality M1 (and M2).

Coloring processes on keratin fibers typically take place in an alkaline environment. To treat keratin fibers and the skin as well as gently as possible, setting a too high pH is not desirable, however. It is preferred, therefore, if the pH of the ready-to-use agent (i.e., the mixture of preparations (a) and (b)) is between 7 and 11 and particularly between 8 and 10.5. pH values within the meaning of the present invention are pH values measured at a temperature of 22° C.

In this case, preparation (a) is preferably adjusted to an alkaline pH, and preparation (b) advantageously has an acidic pH.

In another especially preferred embodiment, a product of the invention is characterized in that

preparation (a) has a pH of 8 to 12.5, preferably of 8.5 to 12, more preferably of 9 to 11.5, and particularly preferably of 9.5 to 11 and

preparation (b) has a pH of 1 to 7, preferably of 1.5 to 6, more preferably of 2.5 to 5.5, and particularly preferably of 3.0 to 5.0.

In another especially preferred embodiment, a product of the invention is characterized in that

preparation (a) has a pH of 8.5 to 12 and

preparation (b) has a pH of 1.5 to 6.

In another especially preferred embodiment, a product of the invention is characterized in that

preparation (a) has a pH of 9 to 11.5 and

preparation (b) has a pH of 2.5 to 5.5.

In another especially preferred embodiment, a product of the invention is characterized in that

preparation (a) has a pH of 9.5 to 11 and

preparation (b) has a pH of 3.0 to 5.0.

Hydrogen peroxide is preferably used as an aqueous solution in preparation (b). The concentration of a hydrogen peroxide solution in the agent of the invention is determined, on the one hand, by legal requirements and, on the other, by the desired effect; preferably, 6 to 12% by weight solutions in water are used. Preparations (b) preferred according to the invention are characterized in that, based on the total weight of preparation (b), they include 0.5 to 20% by weight, preferably 1 to 12.5% by weight of hydrogen peroxide, based on the total weight of the agent.

Preparations (a) and (b) furthermore can include additional active substances, auxiliary substances, and additives in order to improve the coloring or lightening performance and to set other desired properties of the agents.

It has proven advantageous, if the dyes (i.e., preparations (a) and particularly (b)), particularly if they include additional hydrogen peroxide, include at least one stabilizer or complexing agent. Especially preferred stabilizers are phenacetin, alkali benzoates (sodium benzoate), salicylic acid, and dipicolinic acid. Furthermore, all complexing agents in the state of the art can be used. Complexing agents preferred according to the invention are nitrogen-including polycarboxylic acids, particularly EDTA and EDDS, and phosphonates, particularly 1-hydroxyethane-1,1-diphosphonate (HEDP), and/or ethylenediamine tetramethylene phosphonate (EDTMP), and/or diethylenetriamine pentamethylene phosphonate (DTPMP), or sodium salts thereof.

All ionic complexing agents included in preparations (a) and (b) fall in the group of organic salts, and their employed concentrations must therefore be considered in calculating the total molalities M1 or M2.

Further, the preparations of the invention can include other active substances, auxiliary substances, and additives such as, for example, nonionic polymers such as, for example, vinylpyrrolidinone/vinyl acrylate copolymers, polyvinylpyrrolidinone, vinylpyrrolidinone/vinyl acetate copolymers, polyethylene glycols, and polysiloxanes; additional silicones such as volatile or nonvolatile, straight-chain, branched or cyclic, crosslinked or noncrosslinked polyalkylsiloxanes (such as dimethicones or cyclomethicones), polyarylsiloxanes, and/or polyalkylarylsiloxanes, particularly polysiloxanes with organofunctional groups, such as substituted or unsubstituted amines (amodimethicones), carboxy, alkoxy, and/or hydroxyl groups (dimethicone copolyols), linear polysiloxanes(A)-polyoxyalkylene(B) block copolymers, grafted silicone polymers; cationic polymers such as quaternized cellulose ethers, polysiloxanes with quaternary groups, dimethyldiallylammonium chloride polymers, acrylamide-dimethyldiallylammonium chloride copolymers, dimethylaminoethylmethacrylate-vinylpyrrolidinone copolymers quaternized with diethylsulfate, vinylpyrrolidinone-imidazolinium-methochloride copolymers, and quaternized polyvinyl alcohol; zwitterionic and amphoteric polymers; anionic polymers such as, for example, polyacrylic acid or crosslinked polyacrylic acid; structurants such as glucose, maleic acid, and lactic acid, hair-conditioning compounds such as phospholipids, for example, lecithin and kephalins; perfume oils, dimethyl isosorbide, and cyclodextrins; fiber-structure-improving active substances, particularly mono-, di-, and oligosaccharides such as, for example, glucose, galactose, fructose, fruit sugar, and lactose; dyes for coloring the agent; antidandruff agents such as piroctone olamine, zinc omadine, and climbazole; amino acids and oligopeptides; protein hydrolysates with an animal and/or vegetable base, and in the form of their fatty acid condensation products or optionally anionically or cationically modified derivatives; vegetable oils; light stabilizers and UV blockers; active substances such as panthenol, pantothenic acid, pantolactone, allantoin, pyrrolidinone carboxylic acids, and salts thereof, as well as bisabolol; polyphenols, particularly hydroxycinnamic acids, 6,7-dihydroxycoumarins, hydroxybenzoic acids, catechins, tannins, leukoanthocyanidins, anthocyanidins, flavanones, flavones, and flavonols; ceramides or pseudoceramides; vitamins, provitamins, and vitamin precursors; plant extracts; fats and waxes such as fatty alcohols, beeswax, montan wax, and paraffins; swelling and penetration agents such as glycerol, propylene glycol monoethyl ether, carbonates, hydrogen carbonates, guanidines, ureas, and primary, secondary, and tertiary phosphates; opacifiers such as latex, styrene/PVP and styrene/acrylamide copolymers; pearlescent agents such as ethylene glycol mono- and distearate and PEG-3 distearate; pigments and propellants such as propane-butane mixtures, N₂O, dimethyl ether, CO₂, and air.

The selection of these additional substances is made by the skilled artisan according to the desired properties of preparations (a) and (b). In regard to other facultative components and the employed amounts of said components, reference is made expressly to relevant handbooks known to the skilled artisan. The additional active and auxiliary substances are used in the agents of the invention preferably in each case in amounts of 0.0001 to 25% by weight, particularly of 0.0005 to 15% by weight, based on the total weight of the application mixture.

The dispenser within the meaning of the present invention is understood to mean a container include at least two chambers ((A) and (B)), which can be fabricated, for example, from plastic, metal, and/or plastic-metal composites. The dispenser can have, inter alia, the shape of a box, a bottle, a container, a pressurized container, or a tube. Suitable for the product of the invention are all dispenser designs that assure that the product compositions (a) and (b) present in both chambers of the dispenser are kept reliably isolated from another until removal.

Preferably, the individual product compositions are combined to form a total formulation during the product applications, in order to exploit in this way the full effectiveness of the total formulation and to make product use easier in general. For this purpose, the dispenser expediently comprises, in addition to chambers (A), (B), a dispensing head within which the product compositions (a), (b) are conveyed from the chambers to outlet opening (C). In this regard, a suitable mixing device is formed in the dispensing head; upstream from the outlet opening (C), said device provides for the desired mixing of the product compositions (a), (b), before the mixed total formulation, i.e., (a)+(b), is dispensed via outlet opening (C). For example, a generic dispenser with a comparable mixing device is known from DE 3729491 A1, whereby the mixing device therein only has a very short mixing section, however. Such a mixing device according to the invention is structurally integrated directly into the dispensing head or alternatively, however, disposed as a separate structural element within the dispensing head. Static mixers or comparably acting mixing sections, for example, are to be understood to mean suitable mixing devices within the meaning of the invention; these are traversed by the free-flowing product compositions (a), (b) and sufficiently mixed during this through-flow. To this end, such a mixing section typically has suitable flow baffles or flow spoilers, which cause mixing of individual fluid components due to generated turbulences during the surround-flow. Critical for the quality of the mixing of individual fluid components, in this case the product compositions (a), (b), within the mixing device is, inter alia, the selective coordination of the length of the mixing section and the design of the flow baffles to the rheological properties of the product compositions (a), (b). Defined minimum lengths of the mixing section are usually necessary for an adequate mixing result of the mixing device of the invention. Therefore, the mixing section of the mixing device of the invention is preferably designed such that a defined minimum length of the mixing section is guaranteed, without abandoning an overall compact construction of the mixing device and thus of the dispensing head. Thus, adequate mixing results are guaranteed and simultaneously the desired compact outer dimensions of the mixing device and of the dispensing head are assured. To this end, the mixing section is formed, for example, spiral-shaped or comparably compact within the dispensing head.

Chamber (B) of the dispenser can be disposed beside, above, or below chamber (A). Furthermore, chamber (B) can lie within chamber (A) or chamber (A) can lie within chamber (B), whereby in each case the one chamber encloses the other chamber partially or completely. The capacity of chambers (A) and (B) can be 10 cm³ to 1000 cm³ in each case, and the capacity of chambers (A) and (B) can be the same or different. Preferably, the capacity of chambers (A) and (B) is the same. Preferably, chambers A and B are arranged next to one another.

The dispenser has an outlet opening (C), which is in fluid communication with both chambers (A) and (B) or can be brought into fluid communication with it via valves. When the application composition is removed, both formulations (a) and (b) are removed simultaneously via the common outlet opening and are preferably already mixed together upstream by means of the above-described mixing device. The usually reactive individual formulations (a) and (b) thereby come into contact for the first time during product use and hereby form the ready-to-use mixture.

The outlet opening (C) can be, for example, a pressurized container-dispensing device, a pump element, a valve, or a dispensing device for pasty materials. The removal of preparations (a) and (b) via the common outlet opening (C) can occur by pressing the valve or by pumping the pump element.

The dispenser can also be a squeeze container; in this case, preparations (a) and (b) are removed preferably by pressing the squeeze container, whereby the pressure on the squeeze container results in the discharging of preparations (a) and (b) from the common outlet opening (C).

Furthermore, all designs of an outlet opening (C) are also conceivable that enable formulations (a) and (b) to leave the dispenser together.

An especially preferred product is characterized in that

the dispenser is a pressurized container,

the outlet opening (C) is a pressurized container-dispensing device, which also has an appropriate mixing device, and that

the dispenser in addition includes at least one propellant from the group include propane, propene, n-butane, isobutane, isobutene, n-pentane, pentene, isopentane, isopentene, air, nitrogen, argon, N₂O, and/or CO₂.

In the aforementioned preferred embodiment, the product of the invention comprises a pressurized container. Vessels made of metal (aluminum, tin plate, tin), protected or non-splintering plastic or of glass that is coated on the outside with plastic may be used as compressed-gas containers; pressure and breaking strength, corrosion resistance, easy fillability, as well as aesthetic aspects, handling, printability, etc., play a role in their selection. Special protective interior coatings can assure corrosion resistance against the preparation within the pressurized container if necessary.

Very especially preferably, the two chambers (A) and (B) separated from one another are two deformable pouches, preferably made of laminated aluminum, which are each in communication with the outlet opening (C). The outlet opening (C) is a pressurized container-delivery device. Both pouches are in a box-shaped pressurized container, whereby the pressurized container together with the pressurized container-dispensing device close off the dispenser pressure-tight outwardly. The space between the outer wall of the pouch and the inner wall of the pressurized container is filled with at least one propellant. Suitable dispensers are known, for example, from US 2009/0108021 A1. A very especially preferred product for treating keratinic fibers is therefore characterized by such a dispenser with a suitable pressurized container, which has the above-described mixing device. A dispenser constructed in this way takes into account not only the chemical reactivity of the employed individual product compositions (a), (b), but moreover as a result of the mixing provides for a high effectiveness as well of the employed total formulation. Especially good effects according to the invention are achieved when the internal pressure of the pressurized container is at least 1.8 bar, particularly at least 2.5 bar.

The product furthermore comprises a dispensing device (corresponding to outlet opening C), which can have a valve for discharging the application mixture. In a preferred embodiment of the invention, the valve has a valve disc with a coating or a polymeric plastic coating and just such a flexible element with a reset function, which after the operation ends resets the valve to the closed position (=neutral position of the valve). Corresponding cosmetic products in which the aerosol dispensing device comprises a valve, which has a valve cone and/or a flexible element with reset function, which is/are covered with a coating or a polymeric plastic, are also preferred according to the invention.

In another preferred embodiment of the invention, the valve has a flexible element with a reset function and/or a valve disc made of at least one plastic, preferably an elastomeric plastic. Here as well, cosmetic products of the invention in which the valve has a flexible element with a reset function and/or a valve cone made of at least one plastic are preferred, whereby preferred plastics are elastomeric plastics. Especially preferred elastomeric plastics are selected from Buna, particularly Buna N, Buna 421, Buna 1602, and Buna KA 6712, neoprene, butyl, and chlorobutyl.

In another preferred embodiment of the invention, the flexible element with a reset function can be formed as a spiral spring or helical compression spring. In another preferred embodiment of the invention, the flexible element of the valve with the reset function can be formed integrally with the valve cone and have flexible legs. Such a spring can be made of metal or plastic.

All valves used according to the invention preferably have an internally coated valve disc, whereby the coating and valve material are compatible with one another. If aluminum valves are used according to the invention, thus the valve discs thereof can be coated on the inside, e.g., with a Micoflex coating. If tin plate valves are used according to the invention, thus the valve discs thereof can be coated on the inside, e.g., with PET (polyethylene terephthalate). The employed containers, which can be made, e.g., of tin plate or aluminum, aluminum containers being preferred according to the invention, must also be painted or coated on the interior because of the corrosivity of the water-in-oil emulsions, used as taught by the invention.

If the product of the invention is applied via a pressurized container, the dispensers include in addition at least one propellant from the group include propane, propene, n-butane, isobutane, isobutene, n-pentane, pentene, isopentane, isopentene, air, nitrogen, argon, N₂O, and/or CO₂. Within this group, the permanent gases: air, nitrogen, argon, N₂O, and/or CO₂ are preferred; nitrogen, argon, and/or CO₂ are very especially preferred.

Furthermore, it has proven to be preferable if the propellants are also kept at specific pressures in the dispenser. In a preferred embodiment, the dispensers of the invention include one or more additional propellants therefore with a pressure of 3 to 12 bar, preferably of 4 to 10 bar, and especially preferably of 5 to 8 bar, in each case based on the pressure of the propellants between the pouch-shaped chambers (A) and (B) and the pressurized container.

In another especially preferred embodiment, the dispenser is a dispenser for pasty materials, a multi-chamber tube, or a squeeze container. In another very especially preferred embodiment, a product of the invention is characterized in that

the dispenser is a dispenser for pasty materials, a tube, or a squeeze tube and

the outlet opening (C) is a pump element, a valve, or a dispensing device for pasty materials.

The product of the invention according to an alternative design form comprises a multi-chamber tube, which has an inner and outer chamber, both of which end in a common outlet opening (C). The head area is designed such that the two preparations leave the tube together as soon as pressure is exerted on them. The design of said head region determines the pattern in which the preparations leave the tube. In principle, the present invention can comprise any distribution of chambers within the tube. In a first embodiment, for example, the two individual chambers can be arranged next to one another in an outer shell. In an embodiment especially preferred according to the invention, the multi-chamber tube, however, consists of an inner tube completely surrounded by an outer tube.

The multi-chamber tube is preferably fabricated of a material that is suitable for packaging oxidative color changing agents of this type. Laminated aluminum has proven especially suitable according to the invention both for the outer walls and for the inner walls. However, tubes made of plastic laminate (PE, PET, PP) or plastic coextrudates (PE, PET, PP) are also conceivable. Moreover, in one embodiment the material of the inner tube can be selected independently of the material of the outer tube. A tube in which the inner tube is fabricated of an aluminum laminate, which optionally is also protected with a coating, and the outer tube either of aluminum laminate or plastic laminate has proven very especially preferable according to the invention. Aluminum laminate according to the invention is understood to mean a plastic-coated aluminum layer.

It is especially advantageous if the shoulder region of the outer tube is reinforced with round blanks which have especially good barrier properties. In this regard, it is advantageous to incorporate aluminum into the material of the round blanks. To prevent the escape of the mixture during storage and to assure the consumer of the intactness of the tube, it is advantageous to seal the outlet opening with a tamper-evident closure made of aluminum or plastic which is removed by the user.

The selection of the volumes of the individual chambers (A) and (B) is determined by the desired ratio of the volumes of preparation (a) and preparation (b). The volumes of chambers (A) and (B) are preferably the same. The quantity ratio of preparation (a) to the amount of preparation (b) according to the invention is preferably in a range of 1:3 to 3:1; a range of 1:1.5 to 1.5:1 is preferred according to the invention, and a quantity ratio of 1:1 is especially preferred.

Likewise, very especially preferred is a product for the oxidative dyeing and/or lightening of keratinic fibers, include

(1) a dispenser, which

has two chambers (A) and (B) separate from one another,

has an outlet opening (C), which is in communication with chamber (A) and with chamber (B),

(2) a preparation (a) in chamber (A), including in an aqueous cosmetic carrier

one or more thickening polymers in a total amount by weight (G1) of 0.1 to 40% by weight, based on the total weight of preparation (a),

(3) a preparation (b) in chamber (B), including in an aqueous cosmetic carrier

one or more thickening polymers in a total amount by weight (G2) of 0.1 to 40% by weight, based on the total weight of preparation (b), characterized in that

the weight ratio G1/G2 has a value of 0.3 to 3.0 and

the weight ratio of the total weight of preparation (a) to the total weight of preparation (b) is a value of 0.5 to 2.0, preferably of 0.6 to 1.8, more preferably of 0.7 to 1.4, and particularly preferably of 0.8 to 1.2.

The products of the invention can be used in methods for the oxidative changing of hair color. These methods distinguish themselves for the consumer by the particular ease of use, because there is no time-consuming and error-prone preparation of the application mixtures by the consumer. In addition, a particularly uniform color result is enabled with use of the products, because due to the special rheological matching of formulations (a) and (b) it is possible to remove the two formulations in precisely defined proportions, which are always the same relative to one another, during the entire application process.

The removal of the application mixture in this case usually occurs in batches, so that chambers (A) and (B) of the product are emptied stepwise in a defined ratio to one another. The consumer generally removes the amount of the application mixture, necessary for coloring or lightening one or more strands or sections of hair, from the dispenser, and then applies this amount to the hair. This process is repeated by the consumer until either the product has been totally emptied or the consumer has treated all of the hair.

In particular, a continuous removal of the product is also conceivable in the packaging of the product in the form of an pressurized container, however; i.e., in this case chambers (A) and (B) are emptied continuously by the operation of the outlet opening (C), whereby here as well the composition of the application mixture (of preparations (a) and (b)) is the same and defined at each point in time of the removal.

A further subject matter of the present invention is a method for the coloring and/or lightening of keratinic fibers with the use of a product of the first subject matter of the invention, whereby

the dispenser is emptied continuously in one step or in batches in individual steps and

in each step defined, constant amounts of preparations (a) and (b) are removed each time from chambers (A) and (B) via the common outlet opening (C) of the dispenser until both chambers (A) and (B) have been emptied completely.

A method is preferred in which chambers (A) and (B) are emptied in batches in a plurality of steps.

Especially preferred is a method for the coloring and/or lightening of keratinic fibers with the use of a product of the first subject matter of the invention, whereby

the dispenser is emptied in batches in a plurality of steps and

in each step defined, constant amounts of preparations (a) and (b) are removed each time from chambers (A) and (B) via the common outlet opening (C) of the dispenser until both chambers (A) and (B) have been emptied completely.

It can be desirable in this regard that the same amounts of preparations (a) and (b) are removed in each step or each time. If one of chambers (A) or (B) has a larger volume or capacity, however, and/or if one of preparations (a) and (b) is present in a higher amount in the product, thus it can also be desirable to remove preparations (a) and (b) in each step in a constant quantity ratio of 3:1 to 1:3.

It is especially preferred if the amount of preparations (a) and (b), removed in steps, is the same, i.e., if the same amounts (a) and (b) are removed from the dispenser in each removal step.

A method for the coloring and/or lightening of keratinic fibers, therefore, is very especially preferred, which is characterized in that

in each step the same amounts of preparations (a) and (b) are removed each time from chambers (A) and (B) via the common outlet opening (C) of the dispenser until both chambers (A) and (B) have been emptied completely.

The statements made regarding the product of the invention apply mutatis mutandis with respect to other preferred embodiments of the method of the invention.

### Examples

### 1. Preparation of Preparations (a) and (b)

The following preparations (a) and (b) were prepared (all quantities are given in % by weight of active substance):

| Ingredients | | Preparation (a) | Preparation (b) |
|---|---|---|---|
| Xanthan gum | Thickening | 2.00 | 2.00 |
| | polymer | | |
| PEG-60 hydrogenated castor oil | Nonionic | 0.3 | 0.3 |
| | emulsifier | | |
| p-Toluylenediamine, sulfate | ODP | 0.111 | — |
| (C₇H₁₂N₂²⁺ × SO₄²⁻) | Salt | (5.04 × 10⁻³ | |
| (molar mass = 220.25 g/mol); | | mol/kg) | |
| Resorcinol | ODP | 0.03 | — |
| m-Aminophenol | ODP | 0.002 | — |
| 4-Chlororesorcinol | ODP | 0.03 | — |
| 2-Methylresorcinol | ODP | 0.01 | — |
| Ammonium sulfate (NH₄)₂SO₄ | Salt | 0.93 | — |
| (molar mass = 132.13 g/mol) | | (0.070 mol/kg) | |
| Sodium sulfite Na₂SO₃ | Salt | 0.40 | — |
| (molar mass = 126.04 g/mol) | | (0.0317 mol/kg) | |
| Vitamin C | Reducing agent | 0.10 | — |
| Hydroxyethane-1,1- | Salt | — | 0.9 |
| diphosphonic acid, | | | (0.032 mol/kg) |
| dipotassium salt | | | |
| (molar mass = 282.27 g/mol) | | | |
| Ammonia (NH3) | Alkalinizing agent | 2.00 | — |
| Propylene glycol | S | 4.00 | — |
| Dipicolinic acid, | Salt | — | 0.10 |
| monopotassium salt | | | (4.87 × 10⁻³ |
| (molar mass = 205.20 g/mol) | | | mol/kg) |
| Disodium pyrophosphate | Salt | — | 0.03 |
| (H₂O₇P₂Na₂) | | | (1.35 × 10⁻³ |
| (molar mass = 222.053 g/mol) | | | mol/kg) |
| Sodium chloride NaCl | Salt | — | 0.40 |
| (molar mass = 58.44 g/mol) | | | (0.0685 mol/kg) |
| Hydrogen peroxide | OX | — | 6.0 |
| Water (dist.) | | To 100 | To 100 |
| Viscosity (22° C./Brookfield | | 20,000 mPas | 20,000 mPas |
| viscometer/spindle 5/4 rpm), | | | |

Abbreviations:
ODP: oxidation dye precursors
S: solvent
OX: oxidizing agent

### 2. Total Weight Ratio G1/G2

| | Preparation (a) | Preparation (b) |
|---|---|---|
| | G1 | G2 |
| Xanthan gum | 2.00 | 2.00 |

| | |
|---|---|
| Ratio G1/G2 | 1.0 |

### 3. Total Molalities of the Salts in Preparations (a) and (b)

| | Preparation (a) | Preparation (b) |
|---|---|---|
| | M1 | M2 |
| Total molalities salts | 0.107 mol/kg | 0.107 |

| | |
|---|---|
| Ratio M1/M2 | 1.0 |

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A product for the oxidative coloring and/or lightening of keratinic fibers, including
(1) a dispenser, which
has two chambers (A) and (B) separate from one another, and
has an outlet opening (C), which is in communication with chamber (A) and with chamber (B),
(2) a preparation (a) in chamber (A), including in an aqueous cosmetic carrier
one or more thickening polymers in a total amount by weight (G1) of 0.1 to 40% by weight, based on the total weight of preparation (a),
(3) a preparation (b) in chamber (B), including in an aqueous cosmetic carrier
one or more thickening polymers in a total amount by weight (G2) of 0.1 to 40% by weight, based on the total weight of preparation (b),
wherein
the weight ratio G1/G2 has a value of 0.3 to 3.0.

2. The product according to claim 1, wherein the weight ratio of G1/G2 has a value of 0.5 to 2.0.

3. The product according to claim 1, wherein
preparation (a) includes as thickening polymer(s) one or more polymers from the group consisting of polysaccharides, acrylic acid polymers, acrylic acid copolymers, methacrylic acid polymers, and methacrylic acid copolymers, and
preparation (b) includes as thickening polymer(s) one or more polymers from the group consisting of polysaccharides, acrylic acid polymers, acrylic acid copolymers, methacrylic acid polymers, and methacrylic acid copolymers.

4. The product according to claim 1, wherein
preparation (a) includes as thickening polymer(s) one or more polysaccharides from the group consisting of carboxymethylcelluloses, alginic acids, xanthan gum, and the physiologically acceptable salts thereof, and
preparation (b) includes as thickening polymer(s) one or more polysaccharides from the group consisting of carboxymethylcelluloses, alginic acids, xanthan gum, and the physiologically acceptable salts thereof.

5. The product according to claim 1, wherein
preparation (a) includes the thickening polymer(s) in a total amount by weight (G1) of 0.2 to 9.5% by weight based on the total weight of preparation (a), and
preparation (b) includes the thickening polymer(s) in a total amount by weight (G2) of 0.2 to 9.5% by weight based on the total weight of preparation (b).

6. The product according to claim 1, wherein
preparation (a) has a viscosity of 1000 to 100,000 mPas at 22° C. using a Brookfield viscometer/spindle 5/4 rpm,
preparation (b) has a viscosity of 1000 to 100,000 mPas at 22° C. using a Brookfield viscometer/spindle 5/4 rpm, and
the ratio of the viscosities V1/V2 has a value of 0.3 to 3.0.

7. The product according to claim 1, wherein
preparation (a) further includes one or more organic and/or inorganic salts in a total molality (M1) of 0.01 to 1 mol/kg, based on the total weight of preparation (a),
preparation (b) further includes one or more organic and/or inorganic salts in a total molality (M2) of 0.01 to 1 mol/kg, based on the total weight of preparation (b), and
the molality ratio M1/M2 has a value of 0.3 to 3.0.

8. The product according to claim 7, wherein the molality ratio M1/M2 has a value of 0.4 to 2.5.

9. The product according to claim 1, wherein
preparation (a) includes one or more organic and/or inorganic salts in an ionic strength (I1), calculated according to the formula (1)
$$I 1 = \frac{1}{2} {\sum }_{i} {z}_{i}^{2} ci \qquad ( 1 )$$
where zi is the ionic charge of each ion (i) in preparation (a) and
ci is the molality (mol/kg) of each ion in preparation (a), whereby the molality is based on the total weight of preparation (a),
preparation (b) includes one or more organic and/or inorganic salts in an ionic strength (I2), calculated according to the formula (2)
$$I 2 = \frac{1}{2} {\sum }_{i} {z}_{i}^{2} ci \qquad ( 2 )$$
where zi is the ionic charge of each ion (i) in preparation (b) and
ci is the molality (mol/kg) of each ion in preparation (b), whereby the molality is based on the total weight of preparation (b), and
the ratio of the ionic strengths I1/I2 has a value of 0.5 to 2.0.

10. The product according to claim 1, wherein
preparation (a) includes at least one alkalinizing agent, and
preparation (b) includes at least one oxidizing agent.

11. The product according to claim 1, wherein
preparation (a) has a pH of 8 to 12.5, and
preparation (b) has a pH of 1 to 7.

12. The product according to claim 1, wherein
the dispenser is a pressurized container,
outlet opening (C) is a pressurized container-dispensing device, and
the dispenser in addition includes at least one propellant from the group consisting of propane, propene, n-butane, isobutane, isobutene, n-pentane, pentene, isopentane, isopentene, air, nitrogen, argon, N₂O, and/or CO₂.

13. The product according to claim 1, wherein
the dispenser is a dispenser for pasty materials, a tube, or a squeeze tube and
the outlet opening (C) is a pump element, a valve, or a dispensing device for pasty materials.

14. A method for the coloring and/or lightening of keratinic fibers with the use of a product according to claim 1, the method including
emptying the dispenser continuously in one step or in batches in individual steps such that in each step constant amounts of preparations (a) and (b) are removed each time from chambers (A) and (B) via the common outlet opening (C) of the dispenser until both chambers (A) and (B) have been emptied completely.

15. The method according to claim 14, wherein
in each step the same amounts of preparations (a) and (b) are removed each time from chambers (A) and (B) via the common outlet opening (C) of the dispenser until both chambers (A) and (B) have been emptied completely.
